(19) United States
(12) Patent Application Publication
(10) Pub. No.: US 2025/0387488 A1
(43) Pub. Date: Dec. 25, 2025

(54) ACTIVE AGENT-ELUTING HEMOSTATIC AGENTS AND METHODS OF USE THEREOF

(71) Applicant: Florida Southern College, Lakeland, FL (US)

(72) Inventors: Shameka J. Shelby, Auburndale, FL (US); Zachary D. Fralish, Niceville, FL (US); Keith R. Berend, New Albany, OH (US)

(21) Appl. No.: 19/292,654

(22) Filed: Aug. 6, 2025

Related U.S. Application Data
(63) Continuation-in-part of application No. 17/242,219, filed on Apr. 27, 2021, now Pat. No. 12,397,083.
(60) Provisional application No. 63/016,621, filed on Apr. 28, 2020.

(51) Int. Cl. A61K 47/42; A61K 31/192; A61K 31/235; A61K 38/14; A61K 45/06; A61K 47/36; A61P 35/00

(52) U.S. Cl. CPC A61K 47/42; A61K 31/192; A61K 31/235; A61K 38/14; A61K 45/06; A61K 47/36; A61P 35/00

(57) ABSTRACT

A novel active agent-eluting hemostatic agent and methods of use and manufacture thereof are presented. A polymer, such as gelatin or chitosan, is used as the base for a hemostatic agent. The polymer is crosslinked with a chemical crosslinker, such as a carbodiimide, in the presence of an active agent. The active agent may be an anesthetic, chemotherapeutic agent, or antimicrobial, such as an antibiotic. The novel process allows for the active agent to be both covalently bound to the polymer as well as be trapped within cages in the polymer that are formed from the crosslinking. This dual measure allows for controlled and sustained release of the active agent from the hemostatic agent to deliver the active agents to the wound sites.

### CROSS REFERENCE TO RELATED APPLICATIONS

This application is a continuation in part of and claims priority to currently pending U.S. patent application Ser. No. 17/242,219, entitled “Active Agent-Eluting Hemostatic Agents for Prevention of Surgical Site Infection”, filed Apr. 27, 2021, which is a nonprovisional of and claims priority to U.S. Provisional Application No. 63/016,621 entitled “Antibiotic-Eluting Agents for Prevention of Surgical Site Infection”, filed Apr. 28, 2020, the contents of each of which are hereby incorporated by reference into this disclosure.

### FIELD OF INVENTION

This invention relates to hemostatic agents. Specifically, the invention provides novel active agent eluting hemostatic agents and methods of use thereof.

### BACKGROUND OF THE INVENTION

Surgical site infections (SSIs) are a major concern in the healthcare industry that lead to lengthened hospital stays, additional surgical procedures, prolonged antibiotic use, and increased patient morbidity. (Herwaldt, L. A.; Cullen, J. J.; Scholz, D.; French, P.; Zimmerman, M. B.; Pfaller, M. A.; Wenzel, R. P.; Perl, T. M., A prospective study of outcomes, healthcare resource utilization, and costs associated with postoperative nosocomial infections. Infect Control Hosp Epidemiol 2006, 27 (12), 1291-8; Pull ter Gunne, A. F.; Cohen, D. B., Incidence, prevalence, and analysis of risk factors for surgical site infection following adult spinal surgery. Spine (Phila Pa 1976) 2009, 34 (13), 1422-8).

Although infections are often remitted, they increase medical costs and reduce functional prognosis of patients after surgery. (Calderone, R. R.; Garland, D. E.; Capen, D. A.; Oster, H., Cost of medical care for postoperative spinal infections. Orthop Clin North Am 1996, 27 (1), 171-82; Petilon, J. M.; Glassman, S. D.; Dimar, J. R.; Carrcon, L. Y., Clinical outcomes after lumbar fusion complicated by deep wound infection: a case-control study. Spine (Phila Pa 1976) 2012, 37 (16), 1370-4; Chen, S. H.; Lee, C. H.; Huang, K. C.; Hsieh, P. H.; Tsai, S. Y., Postoperative wound infection after posterior spinal instrumentation: analysis of long-term treatment outcomes. Eur Spine J 2015, 24 (3), 561-70). It is estimated that SSIs occur during 2% to 13% of spinal surgeries and periprosthetic joint infections will occur in up to 80,000 patients per year in the United States by 2030 creating a cost burden up to $4 billion annually. (Kurtz, S. M.; Lau, E.; Watson, H.; Schmier, J. K.; Parvizi, J., Economic burden of periprosthetic joint infection in the United States. J Arthroplasty 2012, 27 (8 Suppl), 61-5 cl; Parvizi, J.; Pawasarat, I. M.; Azzam, K. A.; Joshi, A.; Hansen, E. N.; Bozic, K. J., Periprosthetic joint infection: the economic impact of methicillin-resistant infections. J Arthroplasty 2010, 25 (6 Suppl), 103-7; Rechtine, G. R.; Bono, P. L.; Cahill, D.; Bolesta, M. J.; Chrin, A. M., Postoperative wound infection after instrumentation of thoracic and lumbar fractures. J Orthop Trauma 2001, 15 (8), 566-9)

Several preventative methods have been considered effective in preventing SSIs including surgical hand preparations, post-discharge surveillance, postponing elective surgeries in the case of an existing infection, and antimicrobial prophylaxis. (Parvizi, J.; Pawasarat, I. M.; Azzam, K. A.; Joshi, A.; Hansen, E. N.; Bozic, K. J., Periprosthetic joint infection: the economic impact of methicillin-resistant infections. J Arthroplasty 2010, 25 (6 Suppl), 103-7; Owens, C. D.; Stoessel, K., Surgical site infections: epidemiology, microbiology, and prevention. J Hosp Infect 2008, 70 Suppl 2, 3-10). Antimicrobial prophylaxis has become standard practice after orthopedic surgery. (Pull ter Gunne, A. F.; Cohen, D. B., Incidence, prevalence, and analysis of risk factors for surgical site infection following adult spinal surgery. Spine (Phila Pa 1976) 2009, 34 (13), 1422-8.; Rechtine, G. R.; Bono, P. L.; Cahill, D.; Bolesta, M. J.; Chrin, A. M., Postoperative wound infection after instrumentation of thoracic and lumbar fractures. J Orthop Trauma 2001, 15 (8), 566-9; Devin, C. J.; Chotai, S.; McGirt, M. J.; Vaccaro, A. R.; Youssef, J. A.; Orndorff, D. G.; Arnold, P. M.; Frempong-Boadu, A. K.; Lieberman, I. H.; Branch, C.; Hedayat, H. S.; Liu, A.; Wang, J. C.; Isaacs, R. E.; Radcliff, K. E.; Patt, J. C.; Archer, K. R., Intrawound Vancomycin Decreases the Risk of Surgical Site Infection After Posterior Spine Surgery: A Multicenter Analysis. Spine (Phila Pa 1976) 2018, 43 (1), 65-71). Cefazolin and other cephalosporins are considered sufficient to be used in antimicrobial prophylaxis to target Staphylococcus aureus. (Epstein, N. E., Preoperative measures to prevent/minimize risk of surgical site infection in spinal surgery. Surg Neurol Int 2018, 9, 251; Noskin, G. A.; Rubin, R. J.; Schentag, J. J.; Kluytmans, J.; Hedblom, E. C.; Jacobson, C.; Smulders, M.; Gemmen, E.; Bharmal, M., National trends in Staphylococcus aureus infection rates: impact on economic burden and mortality over a 6-year period (1998-2003). Clin Infect Dis 2007, 45 (9), 1132-40). However, due to increased rates of MRSA induced SSI, vancomycin and other glycopeptides/lipopeptides including daptomycin have been more widely used.

Alongside antibiotics, hemostatic agents are considered almost mandatory following orthopedic surgery. Gelatin began replacing clips, electrocoagulation, and ligature to obtain hemostasis in the 1940's and has been widely used ever since due to its biodegradability and biocompatibility. (Green, D.; Wong, C. A.; Twardowski, P., Efficacy of hemostatic agents in improving surgical hemostasis. Transfus Med Rev 1996, 10 (3), 171-82; Jenkins, H. P.; Clarke, J. S., Gelatin sponge, a new hemostatic substance; studies on absorbability. Arch Surg 1945, 51, 253-61). Despite variations in compositions and structures across gelatins, consistently high levels of crosslinking throughout gelatins allows them to function as dependable hemostatic agents. (Olsen, D.; Yang, C.; Bodo, M.; Chang, R.; Leigh, S.; Baez, J.; Carmichael, D.; Perala, M.; Hamalainen, E. R.; Jarvinen, M.; Polarek, J., Recombinant collagen and gelatin for drug delivery. Adv Drug Deliv Rev 2003, 55 (12), 1547-67). The high content of amino acids such as glycine, proline, and hydroxyproline function to potentially accelerate the healing of soft tissue. (Tanaka, A.; Nagate, T.; Matsuda, H., Acceleration of wound healing by gelatin film dressings with epidermal growth factor. J Vet Med Sci 2005, 67 (9), 909-13). The highly hydrophilic nature of gelatin allows for drug absorption in the form of a hydrogel and controlled drug release through a degradation or diffusion-controlled mechanism. (Ikada, Y.; Tabata, Y., Protein release from gelatin matrices. Adv Drug Deliv Rev 1998, 31 (3), 287-301).

The current push for antibiotic-eluting devices to proactively prevent infections can be found throughout recent literature. (Gimeno, M.; Pinczowski, P.; Mendoza, G.; Asin, J.; Vazquez, F. J.; Vispe, E.; Garcia-Alvarez, F.; Percz, M.; Santamaria, J.; Arruebo, M.; Lujan, L., Antibiotic-eluting orthopedic device to prevent early implant associated infections: Efficacy, biocompatibility and biodistribution studies in an ovine model. J Biomed Mater Res B Appl Biomater 2018, 106 (5), 1976-1986). Studies have produced gelatin-based bandages, sponges, and hydrogels that release antibiotic over approximately 7 days. (Shefy-Peleg, A. F., M.; Cohen, B.; Zilberman, M., Novel antibiotic-eluting gelatin-alginate soft tissue adhesives for various wound closing applications. International Journal of Polymeric Materials and Polymeric Biomaterials 2014, 63 (14), 699-707; Shukla, A.; Fang, J. C.; Puranam, S.; Hammond, P. T., Release of vancomycin from multilayer coated absorbent gelatin sponges. J Control Release 2012, 157 (1), 64-71). Alternatively, gelatin sponges incorporating varying concentrations of β-tricalcium phosphate ceramic (β-TCP) have been developed to function as a vancomycin sustained-release system in the treatment of chronic osteomyelitis. (Zhou, J.; Fang, T.; Wang, Y.; Dong, J., The controlled release of vancomycin in gelatin/β-TCP composite scaffolds. Journal of biomedical materials research Part A 2012, 100 (9), 2295-2301). Also, many successful surgeries have incorporated antibiotic-infused bone cement during bone replacement surgeries. (Gandhi, R.; Backstein, D.; Zywiel, M. G., Antibiotic-laden Bone Cement in Primary and Revision Hip and Knee Arthroplasty. J Am Acad Orthop Surg 2018, 26 (20), 727-734; Stravinskas, M.; Nilsson, M.; Horstmann, P.; Petersen, M. M.; Tarasevicius, S.; Lidgren, L., Antibiotic Containing Bone Substitute in Major Hip Surgery: A Long Term Gentamicin Elution Study. J Bone Jt Infect 2018, 3 (2), 68-72). Treatments with bone cement were assessed as efficacious and well tolerated for all patients, indicating the effectiveness of the combination of antibiotics with internal agents. (Kendoff, D. O.; Gehrke, T.; Stangenberg, P.; Frommelt, L.; Bosebeck, H., Bioavailability of gentamicin and vancomycin released from an antibiotic containing bone cement in patients undergoing a septic one-stage total hip arthroplasty (THA) revision: a monocentric open clinical trial. Hip Int 2016, 26 (1), 90-6).

In addition to bioadhesive applications, gelatin conjugates have been studied as anti-cancer agents. Protocols have been established for conjugating doxorubicin and methotrexate to gelatin. (Ofner, C. M., 3rd; Pica, K.; Bowman, B. J.; Chen, C. S., Growth inhibition, drug load, and degradation studies of gelatin/methotrexate conjugates. Int J Pharm 2006, 308 (1-2), 90-9; Kosasih, A. B., B. J.; Wigent, R. J.; & Ofner III, C. M., Characterization and in vitro release of methotrexate from gelatin/methotrexate conjugates formed using different preparation variables. International Journal of Pharmaceutics 2000, 204 (1), 81-89; Cammarata, C. R.; Hughes, M. E.; Ofner, C. M., 3rd, Carbodiimide induced crosslinking, ligand addition, and degradation in gelatin. Mol Pharm 2015, 12 (3), 783-93). Utilizing 1-ethyl-3-(diaminopropyl) carbodiimide HCl (EDC) as a carboxyl activating agent in peptide bond formation, methotrexate was successfully conjugated to gelatin of various molecular weights. EDC-catalyzed conjugations and crosslinking reactions produce an amide bond between carboxyl and amino moieties through a carboxylic anhydride mechanism recognized to occur extensively under aqueous conditions. (Nakajima, N.; Ikada, Y., Mechanism of amide formation by carbodiimide for bioconjugation in aqueous media. Bioconjug Chem 1995, 6 (1), 123-30). These examples exemplify the simplicity and efficiency of peptide bond formation in direct conjugation of small molecules to gelatin.

To limit the prevalence of SSIs, there is a great need to produce highly effective and biocompatible antimicrobial prophylaxis. Effectively combining antibiotics and hemostatic agents should increase antimicrobial presence within surgical sites and improve the efficacy of antimicrobial prophylaxis. In addition, local application can reduce unnecessary systemic administration. This prevents the death of beneficial bacteria within patients and limits selection for antibiotic-resistant bacteria through the reduction of antibiotic application area. Non-steroidal anti-inflammatory drugs (NSAIDs) can also be conjugated to the hemostatic agents herein to provide an anti-inflammatory and anesthetic effect after surgery. Accordingly, what is needed is a biodegradable hemostatic agent that is capable of both immediate and sustained release of antibiotics and other active agents.

### SUMMARY OF INVENTION

The inventors have combined antibiotics with gelatin or chitosan through peptide bond formation via EDC to form an active agent eluting hemostatic agent. This approach allowed for the direct conjugation of antibiotics to gelatin or chitosan in addition to trapping of these antibiotics within crosslinked gelatin or chitosan cages for use as active agent releasing hemostatic agents.

The glycopeptide vancomycin, the lipopeptide daptomycin, the cephalosporins ceftazidime and ceftibuten, the fluoroquinolone ciprofloxacin, the NSAIDs diclofenac and ketorolac, and chemotherapeutics 5FU, doxorubicin, and curcumin were tested in crosslinked gelatin matrices forming the hemostatic agent. The antibiotics ampicillin and ciprofloxacin were tested in crosslinked chitosan matrices forming the hemostatic agent. Vancomycin was tested in chitosan-alginate matrices with varying ratios of the two polymers. Release profiles were analyzed from samples of various reactant ratios to optimize reaction conditions and active agent activity and structural integrity of eluted active agents was confirmed. The biocompatibility and structural makeup of the conjugations were additionally determined.

The inventors found that they were able to produce crosslinked gelatin hemostatic agents in which the active agent, such as antibiotics and anesthetics such as NSAIDs, was both bound to the gelatin itself as well as being trapped within cages formed in the crosslinked gelatin. The “free” antibiotic or NSAID that is in the cages released quicker than the antibiotic that is covalently bound to the gelatin itself thus allowing for a controlled sustained release of antibiotic over a period of at least 3 weeks and controlled release of the NSAID for at least 2 weeks.

In an embodiment, an active agent-eluting hemostatic agent is presented comprising at least one active agent conjugated to and entrapped within cages formed in a crosslinked gelatin. The active agent has at least one amine or carboxylate group in its structure and may be conjugated to the crosslinked gelatin by amide bond formation between the active agent and the gelatin. The hemostatic agent achieves both immediate and controlled sustained release of the active agent. The gelatin may be crosslinked by a chemical crosslinker selected from the group consisting of 1-ethyl-3-(3-dimethylaminopropyl) carbodiimide (EDC) and dicyclohexylcarbodiimide (DCC), and carbonyldiimidazole (CDI). In some embodiments, the crosslinking agent is EDC. The hemostatic agent is a macroscopic product that is not a microparticle or nanoparticle or any other particle having a shell and inner space.

The at least one active agent may be an anesthetic, an antimicrobial, or combinations thereof. In embodiments in which the at least one active agent is an antimicrobial, the antimicrobial may be an antibiotic selected from the group consisting of glycopeptide antibiotics, lipopeptide antibiotics, quinolones, and cephalosporins. Specifically, the antibiotic may be selected from the group consisting of vancomycin, daptomycin, ciprofloxacin, ceftazidime, and ceftibuten.

In alternate embodiments, the at least one active agent may be an anesthetic such as a non-steroidal anti-inflammatory drug (NSAID). The NSAID may be selected from the group consisting of aspirin, ibuprofen, naproxen and naproxen sodium, diclofenac, oxaprozin, etodolac, indomethacin, ketorolac, and vimovo.

In a further embodiment, a method of preventing surgical site infection is presented comprising applying a therapeutically effective amount of an antimicrobial-eluting hemostatic agent to the surgical site. The antimicrobial-eluting hemostatic agent may comprise an antimicrobial conjugated to and entrapped within cages formed in a crosslinked gelatin. The antimicrobial has at least one amine or carboxylate group in its structure and may be conjugated to the crosslinked gelatin by amide bond formation between the antimicrobial and the gelatin. The hemostatic agent releases the antimicrobial in cages first followed by controlled sustained release over a period of time of the antimicrobial conjugated to the gelatin to prevent surgical site infection. The gelatin may be crosslinked by a chemical crosslinker selected from the group consisting of 1-ethyl-3-(3-dimethylaminopropyl) carbodiimide (EDC) and dicyclohexylcarbodiimide (DCC), and carbonyldiimidazole (CDI). In some embodiments, the crosslinking agent is EDC. The hemostatic agent is a macroscopic product that is not a microparticle or nanoparticle or any other particle having a shell and inner space.

In some embodiments, the antimicrobial is an antibiotic selected from the group consisting of glycopeptide antibiotics, lipopeptide antibiotics, quinolones, and cephalosporins. Specifically, the antibiotic may be selected from the group consisting of vancomycin, daptomycin, ciprofloxacin, ceftazidime, and ceftibuten. The antibiotic may be released over a period of about 3 weeks.

In a further embodiment, a method of manufacturing an active agent-eluting hemostatic agent is presented comprising: preparing a solution of gelatin; isolating a carboxyl group concentration from the gelatin solution; incubating at least one active agent, having at least one amine or carboxylate group in its structure, and a crosslinking agent with the isolated carboxyl group concentration for between about 1 hour to about 24 hours to form a product; and precipitating the product to form the active agent-eluting hemostatic agent. The gelatin may be crosslinked by a chemical crosslinker selected from the group consisting of 1-ethyl-3-(3-dimethylaminopropyl) carbodiimide (EDC) and dicyclohexylcarbodiimide (DCC), and carbonyldiimidazole (CDI). In some embodiments, the crosslinking agent is EDC. The hemostatic agent is a macroscopic product that is not a microparticle or nanoparticle or any other particle having a shell and inner space.

The at least one active agent may be an anesthetic, an antimicrobial or a combination thereof. In embodiments in which the at least one active agent is an antimicrobial, the antimicrobial may be an antibiotic that may be selected from a group consisting of glycopeptide antibiotics, lipopeptide antibiotics, quinolones, and cephalosporins. Specifically, the antibiotic may be selected from the group consisting of vancomycin, daptomycin, ciprofloxacin, ceftazidime, and ceftibuten.

In alternate embodiments, the at least one active agent may be an anesthetic such as a non-steroidal anti-inflammatory drug (NSAID). The NSAID may be selected from the group consisting of aspirin, ibuprofen, naproxen and naproxen sodium, diclofenac, oxaprozin, etodolac, indomethacin, ketorolac, and vimovo.

In a further embodiment, an active agent eluting hemostatic agent capable of immediate and sustained release of the active agent is presented comprising: a first amount of at least one active agent, having at least one amine or carboxylate group in its structure, directly conjugated to a crosslinked polymer; and a second amount of the at least one active agent entrapped within cages formed in the crosslinked polymer wherein the polymer is crosslinked to itself via peptide bonds to form the cages. Sustained release may occur over at least 2 weeks. The hemostatic agent is a macroscopic product that is not a microparticle or nanoparticle or any other particle having a shell and inner space.

In some aspects, the crosslinked polymer may be gelatin or chitosan with the active agent eluting hemostatic agent not containing any additional polymers other than gelatin or chitosan. In some aspects, gelatin is the only polymer comprising the active agent eluting hemostatic agent. In other aspects, chitosan is the only polymer comprising the active agent eluting hemostatic agent. In further aspects, a combination of chitosan and alginate are the only polymers comprising the active agent eluting hemostatic agent.

The polymer is crosslinked by the crosslinking agent selected from the group consisting of 1-ethyl-3-(3-dimethylaminopropyl) carbodiimide (EDC) and dicyclohexylcarbodiimide (DCC), and carbonyldiimidazole (CDI). In some aspects, the chemical crosslinker may be EDC at a concentration between about 10 mM to about 60 mM.

The at least one active agent may an anesthetic, an antimicrobial, or a chemotherapeutic agent. In some aspects the active agent is an antimicrobial, specifically an antibiotic comprising vancomycin, daptomycin, ciprofloxacin, ampicillin, or amoxicillin. In other aspects, the active agent is a chemotherapeutic agent comprising 5-fluorouracil (5FU), doxorubicin, or curcumin. In other aspects, the active agent is an anesthetic, specifically a non-steroidal anti-inflammatory drug (NSAID) comprising aspirin, ibuprofen, naproxen and naproxen sodium, diclofenac, oxaprozin, etodolac, indomethacin, ketorolac, or vimovo.

In another aspect, a method of delivering a chemotherapeutic agent to a patient diagnosed with a skin cancer is presented comprising: applying a therapeutically effective amount of a chemotherapeutic agent eluting hemostatic agent to a tumor site of the skin cancer on the patient, the chemotherapeutic agent eluting hemostatic agent comprising: at least one chemotherapeutic agent, having at least one amine or carboxylate group in its structure, entrapped within cages formed in a crosslinked gelatin; wherein the hemostatic agent releases the at least one chemotherapeutic agent by controlled sustained release over at least two weeks. The hemostatic agent is a macroscopic product that is not a microparticle or nanoparticle or any other particle having a shell and inner space.

The method may further comprise excising the tumor prior to applying the therapeutically effective amount of the chemotherapeutic agent eluting hemostatic agent to the tumor site.

The at least one chemotherapeutic agent may be 5-fluorouracil (5FU), doxorubicin, or curcumin. In some aspects, the at least one chemotherapeutic agent is curcumin which has been dissolved prior to crosslinking. The skin cancer may be squamous cell carcinoma (SCC), basal cell carcinoma (BCC), melanoma, or Merkel-cell carcinoma. In some aspects, the skin cancer is SCC.

The gelatin may be crosslinked by a chemical crosslinker selected from the group consisting of 1-ethyl-3-(3-dimethylaminopropyl) carbodiimide (EDC), dicyclohexylcarbodiimide (DCC), and carbonyldiimidazole (CDI). In some aspects, the chemical crosslinker is EDC at a concentration between about 20 mM to about 60 mM.

The chemotherapeutic agent eluting hemostatic agent may be produced by the process comprising: preparing a gelatin solution comprising gelatin type B and a buffer; isolating a portion of the gelatin solution having a carboxyl group concentration of about 20 mM; incubating the chemotherapeutic agent and a crosslinking agent with the portion of the gelatin solution having the isolated carboxyl group concentration to allow complete crosslinking to form a product; subsequently precipitating, washing, drying, and heating the product to form the chemotherapeutic agent eluting hemostatic agent.

In a further aspect, a method of inhibiting an infection in a surgical site of a patient is presented comprising: applying a therapeutically effective amount of an antimicrobial-eluting hemostatic agent to the surgical site, the antimicrobial eluting hemostatic agent comprising: a first amount of at least one antimicrobial, having at least one amine or carboxylate group in its structure, conjugated directly to a crosslinked chitosan matrix via covalent bond; and a second amount of the at least one antimicrobial entrapped within cages formed in the crosslinked chitosan matrix wherein the hemostatic agent releases a portion of the at least one antimicrobial immediately and releases remaining portion by controlled sustained release over at least two weeks. The hemostatic agent is not a microparticle or nanoparticle. In an aspect, the surgical site is an oral surgical site.

The antimicrobial eluting hemostatic agent is produced by a process comprising: preparing a chitosan solution in which solubility is enhanced to lower pH of the chitosan solution; dissolving the antimicrobial in buffer to form an antimicrobial solution; combining equal amounts of the chitosan solution and the antimicrobial solution to form a mixture; subsequently adding an amount of a crosslinking agent to the mixture; agitating the mixture to facilitate zero-length crosslinking; and precipitating, centrifuging, and drying the mixture to form the antimicrobial eluting hemostatic agent.

In an aspect, antimicrobial eluting hemostatic agent may further comprise an amount of alginate to form a chitosan-alginate matrix. The alginate may be present in an amount lower than the amount of chitosan present in the composition.

The chitosan may be crosslinked by a chemical crosslinker selected from the group consisting of 1-ethyl-3-(3-dimethylaminopropyl) carbodiimide (EDC), dicyclohexylcarbodiimide (DCC), and carbonyldiimidazole (CDI). In some aspects, the chemical crosslinker is EDC at a concentration between about 10 mM to about 75 mM.

The antimicrobial may be vancomycin, daptomycin, ciprofloxacin, ampicillin, or amoxicillin.

In an aspect, a method of treating skin cancer in a patient in need thereof is presented comprising: applying a therapeutically effective amount of a chemotherapeutic agent eluting hemostatic agent to a tumor site of the skin cancer on the patient, the chemotherapeutic agent eluting hemostatic agent comprising: at least one chemotherapeutic agent, having at least one amine or carboxylate group in its structure, entrapped within cages formed in a crosslinked gelatin; wherein the hemostatic agent acts as a matrix/scaffold that releases the at least one chemotherapeutic agent by controlled sustained release over at least two weeks. In some aspects, the treatment mitigates recurrence through sustained therapeutic exposure to the chemotherapeutic agent. The hemostatic agent is a macroscopic product that is not a microparticle or nanoparticle or any other particle having a shell and inner space.

The method may further comprise excising the tumor prior to applying the therapeutically effective amount of the chemotherapeutic agent eluting hemostatic agent to the tumor site.

The at least one chemotherapeutic agent may be 5-fluorouracil (5FU), doxorubicin, or curcumin. In some aspects, the at least one chemotherapeutic agent is curcumin which has been dissolved prior to crosslinking. The skin cancer may be squamous cell carcinoma (SCC), basal cell carcinoma (BCC), melanoma, or Merkel-cell carcinoma. In some aspects, the skin cancer is SCC.

The gelatin may be crosslinked by a chemical crosslinker selected from the group consisting of 1-ethyl-3-(3-dimethylaminopropyl) carbodiimide (EDC), dicyclohexylcarbodiimide (DCC), and carbonyldiimidazole (CDI). In some aspects, the chemical crosslinker is EDC at a concentration between about 20 mM to about 60 mM.

In an aspect, a method of preventing recurrence of skin cancer in a patient in need thereof is presented comprising: applying a therapeutically effective amount of a chemotherapeutic agent eluting hemostatic agent to a tumor site of the skin cancer on the patient, the chemotherapeutic agent eluting hemostatic agent comprising: at least one chemotherapeutic agent, having at least one amine or carboxylate group in its structure, entrapped within cages formed in a crosslinked gelatin; wherein the hemostatic agent acts as a matrix/scaffold that releases the at least one chemotherapeutic agent by controlled sustained release over at least two weeks. Recurrence is prevented through sustained therapeutic exposure to the chemotherapeutic agent. The hemostatic agent is a macroscopic product that is not a microparticle or nanoparticle or any other particle having a shell and inner space.

The method may further comprise excising the tumor prior to applying the therapeutically effective amount of the chemotherapeutic agent eluting hemostatic agent to the tumor site.

The at least one chemotherapeutic agent may be 5-fluorouracil (5FU), doxorubicin, or curcumin. In some aspects, the at least one chemotherapeutic agent is curcumin which has been dissolved prior to crosslinking. The skin cancer may be squamous cell carcinoma (SCC), basal cell carcinoma (BCC), melanoma, or Merkel-cell carcinoma. In some aspects, the skin cancer is SCC.

The gelatin may be crosslinked by a chemical crosslinker selected from the group consisting of 1-ethyl-3-(3-dimethylaminopropyl) carbodiimide (EDC), dicyclohexylcarbodiimide (DCC), and carbonyldiimidazole (CDI). In some aspects, the chemical crosslinker is EDC at a concentration between about 20 mM to about 60 mM.

### BRIEF DESCRIPTION OF THE DRAWINGS

For a fuller understanding of the invention, reference should be made to the following detailed description, taken in connection with the accompanying drawings, in which:

FIG. 1A-B are a series of images depicting schematics of gelatin conjugation with vancomycin and daptomycin. Gelatin structure is simplified to show representative carboxylates or amines that may form peptide bonds with the antibiotic of interest when conjugated in the presence of EDC. (A) Carboxylates (shown in grey) on the antibiotics that may form a peptide bond with amine groups in gelatin. (B) Amines (shown in grey) on the antibiotics that may form a peptide bond with carboxylate groups in gelatin.

FIG. 2A-D are a series of images depicting SEM micrographs of antibiotic-releasing hemostatic agents. (A & B) Vancomycin hemostatic agents at 270× and 450× magnifications, respectively. (C & D) Daptomycin hemostatic agents at 270× and 500× magnifications, respectively.

FIG. 3 is a graph depicting the swelling degree of antibiotic-eluting hemostatic agents. Each preparation was loaded with indicated antibiotic concentrations and allowed to swell for 24 hours. 0 mg of antibiotic used buffer in place of antibiotic for total volume and are reflective of crosslinked gelatin only.

FIG. 4A-D are a series of graphs depicting in vitro release of antibiotic from antibiotic-eluting hemostatic agents. Each preparation was loaded with indicated antibiotic concentrations. (A & C) Cumulative release (mg) of vancomycin and daptomycin, respectively. (B & D) Cumulative release (%) of total antibiotic loaded in the hemostatic agent for vancomycin and daptomycin, respectively.

FIG. 5A-B is a series of images depicting FTIR spectra of antibiotic released from hemostatic agents. Pure vancomycin or daptomycin standards were compared to samples released from hemostatic agents. Pure vancomycin (A) or daptomycin (B); both in dark grey as compared to samples released from the hemostatic agents after 2 weeks; both in light grey.

FIG. 6A-C are a series of images depicting bacterial inhibition by vancomycin released from hemostatic agents. (A) Cultured S. aureus was subjected to vancomycin released from either 48 h samples or 2-week samples of antibiotic-eluting hemostatic agent. Dilution 1 contains 9.875 μg/mL for 48 h or 18.25 μg/mL vancomycin in the 2-week samples. Subsequent dilutions reflect a 50% reduction in concentration of the previous dilution. (B) Agar coated with S. aureus exposed to vancomycin-eluting hemostatic agent (1), crosslinked gelatin (2), untreated control disk (3), and 50 μg vancomycin control disk (4). (C) Agar coated with S. aureus exposed to daptomycin-eluting hemostatic agent (1), crosslinked gelatin (2), untreated control disk (3), and 50 μg daptomycin control disk (4).

FIG. 7A-B are a series of graphs depicting normalized cell viability for fibroblasts treated with antibiotic-eluting hemostatic agents. (A) Viability of cells in response to vancomycin-eluting hemostatic agent samples from 48-hour release and controls including equivalent concentration of vancomycin and crosslinked gelatin. (B) Viability of cells in response to daptomycin-eluting hemostatic agent and controls.

FIG. 8 is an image depicting a schematic of gelatin conjugation with ceftibuten and ceftazidime. Gelatin structure is simplified to show representative amines that may form peptide bonds with the antibiotic of interest when conjugated in the presence of EDC. Carboxylates shown in grey on the antibiotics that may form a peptide bond with amine groups in gelatin.

FIG. 9 is an image depicting a schematic of gelatin conjugation with ciprofloxacin. Gelatin structure is simplified to show representative amines that may form peptide bonds with the antibiotic of interest when conjugated in the presence of EDC. Carboxylates shown in grey on the antibiotics that may form a peptide bond with amine groups in gelatin.

FIG. 10A-B is a series of images depicting SEM micrographs of antibiotic-releasing hemostatic agents. Ceftazidime hemostatic agent at 270× (A) and 450× magnifications (B).

FIG. 11 is a graph depicting the swelling degree of antibiotic-eluting hemostatic agents. Each preparation was loaded with indicated antibiotic concentrations and allowed to swell for 24 hours. 0 mg of antibiotic used buffer in place of antibiotic for total volume and are reflective of crosslinked gelatin only.

FIG. 12A-D are a series of graphs depicting in vitro release of antibiotic from antibiotic-eluting hemostatic agents. Each preparation was loaded with indicated antibiotic concentrations. (A & C) Cumulative release (mg) of ciprofloxacin and ceftazidime, respectively. (B & D) Cumulative release (%) of total antibiotic loaded in the hemostatic agent for ciprofloxacin and ceftazidime, respectively.

FIG. 13 is an image depicting FTIR spectra of antibiotic released from hemostatic agents. Pure ceftazidime standard was compared to samples released from hemostatic agents. Pure ceftazidime in dark grey, compared to samples released from the hemostatic agents after 2 weeks in light grey.

FIG. 14A-B are a series of graphs depicting normalized cell viability of fibroblasts in response to ciprofloxacin-eluting hemostatic agent samples (A) and ceftazidime-eluting hemostatic agent samples (B) from 48-hour release and controls including equivalent concentration of ciprofloxacin/ceftazidime and crosslinked gelatin.

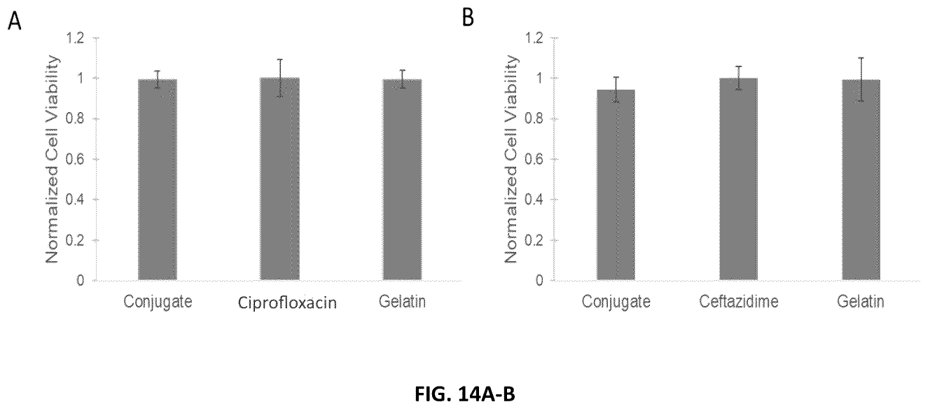

FIG. 15 is a schematic of gelatin conjugation with diclofenac or ketorolac. Gelatin structure is simplified to show representative amines that may form peptide bonds with the NSAID of interest when conjugated in the presence of EDC. The carboxylate groups shown in grey on the diclofenac or ketorolac are those that may form a peptide bond with amine groups in gelatin.

FIG. 16A-D are a series of graphs depicting in vitro release of diclofenac or ketorolac from NSAID-eluting hemostatic agents. Each preparation was loaded with indicated concentrations. (A) Cumulative release (mg) of diclofenac. (B) Cumulative release (%) of total diclofenac loaded in the hemostatic agent. (C) Cumulative release (mg) of ketorolac. (D) Cumulative release (%) of total ketorolac loaded in the hemostatic agent.

FIG. 17 is an image depicting FTIR spectra of ketorolac released from hemostatic agents. Pure ketorolac standards were compared to samples released from hemostatic agents over time. Pure ketorolac as compared to samples released from the hemostatic agents after 72 hours, 1 week, and 2 weeks.

FIG. 18A-B are a series of graphs depicting normalized cell viability for fibroblasts treated with NSAID-eluting hemostatic agents. (A) Viability of cells in response to diclofenac-eluting hemostatic agent samples from 48-hour release and controls including equivalent concentration of diclofenac and cross-linked gelatin. (B) Viability of cells in response to ketorolac-eluting hemostatic agent and controls.

FIG. 19 is a series of images depicting structures of anti-cancer molecules used in the study. Gelatin structure is omitted for simplicity but contains primary amines and carboxylates necessary to create amide bonds in the presence of EDC to create cages. Only doxorubicin contains a primary amine that may directly conjugate to the gelatin.

FIG. 20A-C are a series of graphs depicting in vitro release of anticancer agents from gelatin-based drug-eluting conjugates. Each preparation was loaded with the indicated drug concentration. (A) Cumulative release (mg) of 5-fluorouracil; (B) cumulative release of curcumin; and (C) cumulative release of doxorubicin.

FIG. 21A-C are a series of graphs depicting normalized cell viability for fibroblasts treated with anticancer drug-eluting gelatin conjugates. (A) Viability of cells in response to 5-fluorouracil-eluting conjugate samples from 48-hour release and controls including equivalent concentration of 5FU and cross-linked gelatin. (B) Viability of cells in response to curcumin-eluting conjugate and controls. (C) Viability of cells in response to doxorubicin-eluting conjugate and controls.

FIG. 22A-B are images of the (A) chitosan only conjugate and (B) dried chitosan only conjugate.

FIG. 23 is a series of images depicting a schematic of chitosan conjugation with ciprofloxacin and ampicillin. Chitosan structure is simplified to show representative amines that may form peptide bonds with the antibiotic of interest when conjugated in the presence of EDC. Carboxylates circled on the antibiotics that may form a peptide bond with amine groups circled in chitosan.

FIG. 24A-B are a series of images depicting in vitro release of antibiotic from antibiotic-eluting hemostatic agents. Each preparation was loaded with indicated antibiotic concentrations. (A) Cumulative release (mg) of ciprofloxacin, (B) Cumulative release of ampicillin.

FIG. 25A-B are a series of images depicting bacterial inhibition by antibiotics released from chitosan hemostatic agents. (A) Agar coated with E. coli exposed to ciprofloxacin-eluting hemostatic agent, and (B) Agar coated with E. coli exposed to ampicillin-eluting hemostatic agent.

FIG. 26 is a series of images depicting a schematic of vancomycin, chitosan, and alginate conjugation to show representative carboxylates or amines that may form peptide bonds with the antibiotic of interest when conjugated in the presence of EDC. Carboxylates shown in light grey on vancomycin and alginate that may form a peptide bond with amine groups in chitosan. Amines shown in light grey.

FIG. 27A-D are a series of images depicting in vitro release of antibiotic from antibiotic-eluting hemostatic agents. Each preparation was loaded with indicated antibiotic concentrations. (A) Cumulative release (mg) of vancomycin from complex 1; (B) complex 2; (C) complex 3; (D) complex 4.

FIG. 28 is an image depicting FTIR spectra of vancomycin released from hemostatic agents. Pure vancomycin standards were compared to samples released from hemostatic agents over time. Pure vancomycin as compared to samples released from the complex 1 after 24, 48, and 72 hours.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### Definitions

All numerical designations, including ranges, are approximations which are varied up or down by increments of 1.0 or 0.1, as appropriate. It is to be understood, even if it is not always explicitly stated that all numerical designations are preceded by the term “about.” It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined. As used herein, the term “about” refers to ±10%.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5 but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used in the specification and claims, the singular form “a,” “an” and “the” include plural references unless the context clearly dictates otherwise. For example, the term “a nanoparticle” includes a plurality of nanoparticles, including mixtures thereof.

As used herein, the term “comprising” is intended to mean that the products, compositions, and methods include the referenced components or steps, but not excluding others. “Consisting essentially of” when used to define products, compositions, and methods, shall mean excluding other components or steps of any essential significance. “Consisting of” shall mean excluding more than trace elements of other components or steps.

“Patient” is used to describe an animal, preferably a human, to whom treatment is administered, including prophylactic treatment with the compositions of the present invention.

“Pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pennsylvania, Mack Publishing Company, 19^{th} ed.) describes formulations which can be used in connection with the subject invention.

The terms “administer” or “administering” as used herein are defined as the process by which the compositions of the present invention are delivered to the patient for treatment or prevention purposes. The composition can be delivered topically in dosage unit formulations containing conventional nontoxic pharmaceutically acceptable carriers, adjuvants, and vehicles as desired. The compositions can be administered prior to or after wound closure. Administration may occur once or multiple times.

“Sustained release” as used herein refers to a composition comprising a therapeutically effective amount of the active agent-eluting hemostatic agent, when administered to a patient, continuously releases a stream of one or more active agents over a predetermined time period at a level sufficient to achieve a desired effect, such as preventing or treating infections, inflammation or pain, throughout the predetermined time period. In some aspects, the one or more active agents are released over a period of at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, or longer. In some aspects, the one or more active agents are released over a period of at least 1 week, at least 2 weeks, at least 3 weeks, at least 4 weeks, at least 5 weeks, at least 6 weeks, or longer. Reference to a continuous release stream is intended to encompass release that occurs as the result of biodegradation of the composition, or component thereof, or as the result of metabolic transformation or dissolution of the added nutrients or other desired agents.

“Active agent” as used herein is defined as a substance, component or agent that has measurable specified or selective physiological activity when administered to an individual in a therapeutically effective amount. Examples of active agents as used in the present invention include anesthetics and antimicrobials such as antibiotics, antivirals, antifungals, antiprotozoals, and antiparasitics. At least one active agent is used in the compositions of the present invention. The active agent should have at least one amine or carboxylate group in its structure to ensure binding with the hemostatic agent. In some aspects, the active agent is a chemotherapeutic drug useful to treat skin cancer. Examples of chemotherapeutic drugs useful in the instant invention include, but are not limited to, 5-fluorouracil (5-FU), doxorubicin, curcumin, imiquimod, cisplatin, carboplatin, paclitaxel, dacarbazine, temozolomide, etoposide, topotecan, and derivatives and salts thereof.

With regard to the processes used to form the hemostatic agent described herein, there is an excess amount of active agent molecules in the reactions which allows for an amount of the active agent molecules (a first amount) to be crosslinked and a separate portion of the active agent molecules (a second amount) to be free. Kinetic considerations allow for the gelatin or chitosan to preferentially bind to itself to form cages and to also allow for some of the active agent molecules to directly crosslink to the gelatin or chitosan. This is concluded since reactions with and without active agent molecules added demonstrate the same overall morphology and by the fact that products made with active agent molecules included tend to last longer than those generated without active agent molecules, particularly when the active agent molecules have more available amine and carboxyl groups which suggests that it aids in crosslinking instead of impeding crosslinking. This is shown in the differences in release times with vancomycin/daptomycin versus NSAIDs. In addition, active agent molecules having 2 or more amine or carboxyl groups not only have the ability to crosslink with the gelatin or chitosan but also participate in cage formation with multiple of the active agent molecule's carboxyl/amine groups bonding with a gelatin or chitosan carboxyl/amine group, basically forming a cage comprised of gelatin-active agent molecule-gelatin or chitosan-active agent molecule-chitosan. Excess active agent molecules in these situations can be trapped in the formed “cages” allowing for both immediate and sustained release of the active agent.

“Excess amount” as used herein with respect to active agent molecules, as enumerated in the previous paragraph, refers to the molar ratios of active agent molecules to polymer so as to have sufficient active agent molecules to covalently bind to all of the available groups on the polymer as well as an additional amount of active agent molecules available to be “trapped” in the cages. For example, in the gelatin and diclofenac example below, a sufficient amount of diclofenac must be available to ensure that the one carboxylate each molecule has available can form a peptide bond with the amines available on the gelatin, as well as have additional diclofenac left over to be trapped in the cages. This additional amount would not be capable of forming a peptide bond since all the binding sites on the gelatin are occupied. The numerical “excess amount” will change for each active agent and polymer combination dependent upon the number of amino/carboxyl groups present on the specific active agent and specific polymer. In some aspects, for example, the ratio of active agent to polymer may be 2:1, however this ratio is not intended to be limiting, and other ratios are contemplated for use herein dependent upon the specific active agent and polymer.

“Chemotherapeutic drug” as used herein refers to a drug used to inhibit growth of cancer cells in the body by killing the cancer cells and/or inhibiting cancer cell proliferation. Examples of chemotherapeutic drugs useful in the instant invention include, but are not limited to, 5-fluorouracil (5-FU), doxorubicin, curcumin, imiquimod, cisplatin, carboplatin, paclitaxel, dacarbazine, temozolomide, etoposide, topotecan and derivatives and salts thereof. In some aspects, the concentration of 5-FU may be between about 30 mM to about 80 mM, including all amounts in between. The concentration of doxorubicin may be between about 0.1 mM to about 0.5 mM, including all amounts in between. The concentration of curcumin may be about 500 mM to about 1 M. All concentrations are exemplary and are not to be construed in a limiting way as other concentrations are contemplated.

“Skin cancer” as used herein refers to a pathological condition characterized by the uncontrolled growth of abnormal skin cells, typically resulting from DNA damage, often due to ultraviolet radiation, that triggers mutations. The term encompasses both invasive and in situ forms and may be diagnosed clinically or via histopathology. Examples of skin cancers capable of treatment with the instant invention include, but are not limited to, squamous cell carcinoma (SCC), basal cell carcinoma (BCC), melanoma, and Merkel-cell carcinoma.

A “therapeutically effective amount” as used herein is defined as concentrations or amounts of components which are sufficient to effect beneficial or desired clinical results, including, but not limited to, any one or more of treating symptoms of infection, inflammation, cancer, or pain and preventing infection or cancer recurrence or treating or preventing pain, inflammation, cancer, or infection.

“Prevention” or “preventing” as used herein refers to any of: halting the effects of infection, cancer, or pain, reducing the effects of infection, cancer, or pain, reducing the incidence of infection, cancer, or pain, reducing the development of infection, cancer, or pain, delaying the onset of symptoms of infection, cancer, or pain, increasing the time to onset of symptoms of infection, cancer, or pain, and reducing the risk of development of infection, cancer, or pain.

“Treatment” or “treating” as used herein refers to any of the alleviation, amelioration, elimination, and/or stabilization of a symptom, as well as delay in progression of a symptom of a particular disorder. For example, “treatment” may include any one or more of the following: amelioration and/or elimination of one or more symptoms associated with infection, inflammation, cancer, or pain; reduction of one or more symptoms of infection, inflammation, cancer, or pain; stabilization of symptoms of infection, inflammation, cancer, or pain; and delay in progression of one or more symptoms of infection, inflammation, cancer, or pain.

“Infection” as used herein refers to the invasion of one or more microorganisms such as bacteria, viruses, fungi, yeast, or parasites in the body of a patient in which they are not normally present.

“Antibiotics” as used herein refers to natural or synthetic compositions capable of killing or inhibiting growth of bacteria. Exemplary antibiotics that may be used herein include any antibiotic having at least one amine or carboxylate group in its structure including, but not limited to, glycopeptide antibiotics, lipopeptide antibiotics, cephalosporins, penicillins, and quinolones. Examples of glycopeptide antibiotics include, but are not limited to, vancomycin, teicoplanin, oritavancin, telavancin, and dalbavancin. In some embodiments in which vancomycin is used, the concentration can be between about 0.069 mM to about 2.07 mM, including all amounts in between. Examples of lipopeptide antibiotics include, but are not limited to, daptomycin, surfactin, iturin, fengycin, and polymyxin. In some embodiments in which daptomycin is used, the concentration can be between about 0.8 mM to about 1.85 mM, including all amounts in between. Examples of cephalosporins include, but are not limited to, ceftazidime, ceftibuten, cefazolin, cefaclor, cefdinir, cefuroxime, cefadroxil, cephalexin, cefepime, ceftriaxone, cefotetan, cefoxitin, cefprozil, ceftaxime, cefditoren, cefixime, cefpodoxime, ceftaroline, Cefacetrile (cephacetrile), Cefadroxil (cefadroxyl; Duricef®), Cefalexin (cephalexin; Keflex®), Cefaloglycin (cephaloglycin), Cefalonium (cephalonium), Cefaloridine (cephaloradine), Cefalotin (cephalothin; Keflin®), Cefapirin (cephapirin; Cefadryl®), Cefatrizine, Cefazaflur, Cefazedone, Cefazolin (cephazolin; Ancef®, Kefzol®), Cefradine (cephradine; Velosef®), Cefroxadine, Ceftezole, Cefaclor (Ceclor®, Distaclor®, Keflor®, Raniclor®), Cefonicid (Monocid®), Cefprozil (cefproxil; Cefzil®), Cefuroxime (Altacef®, Zefu®, Zinnat®, Zinacef®, Ceftin®, Biofuroksym®, Xorimax®), Cefuzonam, Cefaloram, Cefmetazole, Cefotetan, Cefoxitin, Loracarbef, Cefbuperazone, Cefmetazole (Zefazone®), Cefminox, Cefotetan (Cefotan®), Cefoxitin (Mefoxin®), Cefotiam (Pansporin®), Cefcapene, Cefdaloxime, Cefdinir (Sefdin®, Zinir®, Omnicef®, Kefnir®), Cefditoren, Cefetamet, Cefixime (Fixx®, Zifi®, Suprax®), Cefmenoxime, Cefodizime, Cefotaxime (Claforan®), Cefovecin (Convenia®), Cefpimizole, Cefpodoxime (Vantin®, PECEF®, Simplicef®), Cefteram, Ceftibuten (Cedax®), Ceftiofur (Naxcel®, Excenel®), Ceftiolene, Ceftizoxime (Cefizox®), Ceftriaxone (Rocephin®), Cefoperazone (Cefobid®), Ceftazidime (Meezat®, Fortum®, Fortaz®), Latamoxef (moxalactam), Cefclidine, Cefepime (Maxipime®), Cefiderocol (Fetroja®), Cefluprenam, Cefoselis, Cefozopran, Cefpirome (Cefrom®), Flomoxef, Ceftobiprole, Ceftaroline, Ceftolozane, Cefaparole, Cefcanel, Cefedrolor, Cefempidone, Cefetrizole, Cefivitril, Cefmatilen, Cefmepidium, Cefoxazole, Cefrotil, Cefsumide, Ceftioxide, Cefuracetime, and Nitrocefin. Exemplary quinolones include, but are not limited to, fluoroquinolones such as ciprofloxacin, levofloxacin, gemifloxacin, and moxifloxacin. In some aspects, the concentration of fluoroquinolones may be between about 0.1 mM to 15 mM. Examples of penicillins include, but are not limited to, amoxicillin, ampicillin, nafcillin, oxacillin, dicloxacillin, piperacillin, carbenicillin, ticarcillin. In some aspects, the concentration of penicillins may be between about 5 mM to 39 mM.

“Antimicrobial” as used herein refers to natural or synthetic compositions capable of killing or inhibiting the growth of microorganisms including, but not limited to, bacteria, fungi, viruses, protozoa, and parasites. Antimicrobials that may be used include those having at least one amine or carboxylate group in its structure. Antimicrobials used herein include antibiotics, antivirals, antifungals, antiprotozoals, and antiparasitics. Exemplary antimicrobials that may be used herein include, but are not limited to, the antibiotics as defined previously, povidone-iodine, iodine, and betadine.

“Anesthetics” as used herein refers to a natural or synthetic composition capable of producing a local, regional, or general loss of sensation. Anesthetics are generally used to induce an insensitivity to pain. The terms “anesthetic” and “analgesic” are used interchangeably herein. Anesthetics that may be used include those having at least one amine or carboxylate group in its structure. As used herein, the term “anesthetic” may refer to local anesthetics. Exemplary local anesthetics that may be used herein include, but are not limited to, lidocaine, marcaine, bupivacaine, prilocaine, mepivacaine, etidocaine, ropivacaine, and levobupivacaine. “Anesthetic” may also refer to compositions exhibiting anti-inflammatory properties such as non-steroidal anti-inflammatory drugs (NSAIDs). Exemplary NSAIDs useful herein include, but are not limited to, aspirin, ibuprofen, naproxen and naproxen sodium, diclofenac, oxaprozin, etodolac, indomethacin, ketorolac, and vimovo.

“Hemostatic agent” as used herein refers to a substance or composition used to stop bleeding, hemorrhage, or blood flow through a vessel or body part. In some embodiments, the hemostatic agent may be a polymer matrix forming a physical barrier over the surgical site or wound. In some aspects, the hemostatic agent is absorbable. In some embodiments, the hemostatic agent has a paste-like consistency. Examples of hemostatic agents include, but are not limited to, gelatin, collagen, chitosan, and derivatives thereof. In a preferred embodiment, crosslinked gelatin is used as the hemostatic agent. The hemostatic agent preferably has a semisolid paste-like consistency that is more solid than a hydrogel but less solid than a sponge. In other embodiments, the hemostatic agent has the consistency of a dry powder which may be clumpy. In other embodiments, a pharmaceutically active carrier is added to the dry powder to achieve a paste-like consistency. The term “conjugate” is used synonymously herein with “hemostatic agent.” Of note, the conjugates formed by the processes described herein do not form, and are incapable of forming, a structure having a shell/wall and an inner core within which the therapeutic agent is encapsulated such as a micro- or nanocarrier. The all-aqueous solution used herein actively works against the generation of enclosed particles such as nanoparticles, as the high molecular weight portions inhibit the formation of nanoparticles. Nanoparticles are known in the art to require minimal crosslinking to allow for the gelatin or other polymer to remain as small particles in solution. For example, the conjugates described herein do not include microparticles, nanoparticles, nanospheres, microspheres, microcapsules, liposomes, micelles, micropellets, microgranules, microsponges, colloidosomes, nanofibers, or dendrimers. The conjugates formed herein are non-spherical macroscopic products, i.e., capable of being seen with the naked eye and having a size of at least 100 microns. Further, the conjugates/hemostatic agents are not equivalent to a bioadhesive, a synthetic “glue” formed from biological monomers designed to adhere to biological tissues. Hemostatic agents are known in the art to facilitate clot formation either through enzymatic reactions or through mechanical compression. In contrast, bioadhesives are designed to adhere to biological tissues and do not assist in the internal cellular signaling that promotes wound healing.

“Bonding structure” as used herein refers to the interaction of the active agent with the polymer to form the hemostatic agent described herein. For gelatin, the active agent bonds directly with the gelatin through covalent bonding and weak interactions such as hydrogen bonding, electrostatic interactions, van der Waals interactions, etc. as well as the gelatin interacting with itself through covalent bond formation. The use of the crosslinker allows for a nucleophilic attack which results in a large, stable leaving group that is removed in the wash steps. Degradation occurs through hydrolysis of the peptide bonds which is energetically favorable to the body as opposed to enzymatic assistance.

“Cage-like” or “cage” as used herein refers to a three-dimensional arrangement that forms a cage-like structure comprised of interconnected crosslinking of a polymer to itself through covalent bond formation or of one polymer bonding to another polymer. The cage structure formed does not form a fully enclosed structure having a continuous shell/wall such as in a microparticle or nanoparticle. In some aspects, the crosslinks form the “bars” of the cage.

“Powder” as used herein refers to a loose, dry, solid substance in a finely divided state. “Powder” as used herein refers to a bulk powder comprised of a plurality of individual powder particles with the bulk powder being visible to the naked eye, i.e., macroscopic.

“Polymer” as used herein refers to a relatively high molecular weight organic compound, natural or synthetic, whose structure can be represented by a repeated small unit, the monomer. Synthetic polymers are typically formed by addition or condensation polymerization of monomers. Examples of polymers that can be used as hemostatic agents include, but are not limited to, gelatin, collagen, and derivatives thereof. Ideally, polymers used herein have primary amines or carboxylates capable of covalent conjugation. In some aspects, gelatin is the only polymer in the composition. In other aspects of the invention, polymers may include, but not be limited to, chitosan, alginate, and combinations thereof. Of note, alginate is not used in combination with gelatin given the propensity of alginate to bind to gelatin in the presence of a crosslinker. Such binding inhibits gelatin from crosslinking with itself to form the “cages” as well as inhibits direct conjugation of the active agent to the gelatin for sustained release since the alginate competes with the active agent for binding to gelatin. In some aspects the conjugate does not contain any additional polymers other than gelatin. In some aspects, the conjugate does not contain any additional polymers other than chitosan. In some aspects, the conjugate contains a combination of only chitosan and alginate as polymers. The carboxyl group concentration of gelatin may be between about 5-20 mM. The carboxyl group concentration will change based on the polymer or combination of polymers used since each will contain varying molar concentrations of carboxyl groups. The alginate will be much smaller since the molecule is smaller and there is a carboxyl in each repeating unit. In an aspect, the amount of alginate may be between about 0.03-0.1 wt. %. The chitosan has no carboxyl groups and thus amine concentration is measured. In some aspects, the amine concentration range for chitosan may be between about 1 mM to about 18 mM. In some aspects, the chitosan may be medium molecular weight chitosan having, for example, a range of concentrations between about 2 mM to about 18 mM, including all amounts in between. In other aspects, the chitosan may be low molecular weight chitosan having, for example, a range of concentrations between about 1 mM to about 6 mM, including all amounts in between.

“Crosslinking” as used herein refers to chemically joining two or more molecules by a covalent bond. As used herein, crosslinking the hemostatic agent, such as gelatin, with a chemical crosslinking agent forms cages in the hemostatic agent. Crosslinking also allows one of more anesthetics or antimicrobials to be covalently bound directly to the gelatin and to be encapsulated in the cages formed in the gelatin. Exemplary crosslinking agents include, but are not limited to, carbodiimides such as 1-ethyl-3-(3-dimethylaminopropyl) carbodiimide (EDC) and dicyclohexylcarbodiimide (DCC); carbonyldiimidazole (CDI). Controlling crosslink density is of vital importance. An overabundance of crosslinks results in a sponge, which is not desirable. While lower levels of crosslinking, in some aspects where the crosslinker is present at lower concentrations, does not allow the cages to form, thus releasing the antibiotic too quickly. Concentrations of the crosslinking agent and the polymer, such as gelatin or chitosan, are controlled to ensure steady elution profiles of the antibiotic. In some aspects, the concentration of chitosan may be between about 2 mM to about 18 mM, including all amounts in between. In some aspects, the chitosan may in an amount from about 1 mg/mL to about 6 mg/mL, including all amounts in between. In some aspects, the concentration of gelatin may be between about 100 mM to about 250 mM, including all amounts in between. In an aspect, the amount of gelatin may be between about 10 mg/mL to about 25 mg/mL, including all amounts in between. In some embodiments in which EDC is used as the crosslinking agent, the concentration can be between about 10 mM to about 75 mM, including all amounts in between. In some aspects, the concentration is about 20 mM to about 50 mM, including all amounts in between. In some aspects, the concentration of EDC is about 10 mM, 15 mM, 20, mM, 25 mM, 30 mM, 35 mM, 40 mM, 45 mM, 50 mM, 55 mM, 60 mM, 65 mM, 70 mM, or 75 mM. In an aspect, the amount of EDC may be between about 9 mg/mL to about 15 mg/mL, including all amounts in between. When the term “complete crosslinking” is used herein, this refers to every available crosslink between the polymer and the active agent has been made.

“Gelatin” as used herein refers to a water-soluble polymer obtained from acid, alkaline or enzymatic hydrolysis of collagen. Gelatin derived from acid treatment is referred to as type A while gelatin derived from alkaline treatment is referred to as type B. In some embodiments, the gelatin may be derived from porcine skin, bovine skin, bone, poultry, or fish. In other embodiments, the gelatin may be a recombinant gelatin. In some embodiments, the gelatin used is Type B gelatin. In some embodiments, the amount of gelatin used may be between about 5 mg/ml to about 20 mg/ml, including all amounts in between. In some aspects, the carboxyl group concentration of gelatin may be between about 5 mM to about 20 mM, including all amounts in between.

“Paste-like” as used herein refers to a semisolid composition capable of being spread over a wound site and is thicker than a hydrogel but not solid like a sponge.

“Semi-solid” as used herein refers to a composition having a somewhat firm consistency with rigidity and viscosity between a solid and a liquid.

“Gel-like” or “jelly-like” as used herein refers to a macroscopic semi-solid or viscoelastic material that exhibits properties intermediate between those of a solid and a liquid, characterized by the ability to maintain its shape under low stress but capable of flowing under higher stress. The material typically displays a yield stress and exhibits a measurable storage modulus (G′) higher than the loss modulus (G″) over a given frequency range. In some aspects the G′/G″ ratio is greater than 1 over a specified frequency range. In some aspects, the conjugates described herein exhibit a jelly-like structure but are not true hydrogels as the chitosan used herein is hydrophilic, as opposed to hydrophobic, since it is deacetylated. During crosslinking, the overall structure of the chitosan and chitosan/alginate conjugates described herein is fairly hydrophilic as it will eventually completely solubilize in water.

“Hydrogel” as used herein refers to a three-dimensional network of hydrophobic polymers synthesized by crosslinking water-soluble polymers. Hydrogels are capable of swelling in water and absorbing and retaining a large amount of water (or biological fluids) while maintaining structural integrity due to physical and/or chemical crosslinking of individual polymer chains. Hydrogels have an elastic, jelly-like structure.

“Sponge” as used herein refers to a solid, water-insoluble, non-elastic, pliable bio-compatible material capable of being used in the body as a hemostatic agent.

The inventors have developed glycopeptide and lipopeptide-eluting hemostatic agents that are suitable in the prevention of SSIs. These antibiotic-eluting hemostatic agents are novel in their approach to long-term release of antibiotics within surgical sites through the use of EDC for gelatin crosslinking in addition to specific conjugation of the antibiotic to the gelatin, yielding a continuous release of antibiotic greater than the minimum inhibitory concentrations (MIC) but lower than toxicity levels. (Kshetry, A. O.; Pant, N. D.; Bhandari, R.; Khatri, S.; Shrestha, K. L.; Upadhaya, S. K.; Poudel, A.; Lekhak, B.; Raghubanshi, B. R., Minimum inhibitory concentration of vancomycin to methicillin resistant Staphylococcus aureus isolated from different clinical samples at a tertiary care hospital in Nepal. Antimicrobial Resistance & Infection Control 2016, 5 (1), 27; Van Hal, S.; Lodise, T. P.; Paterson, D. L., The clinical significance of vancomycin minimum inhibitory concentration in Staphylococcus aureus infections: a systematic review and meta-analysis. Clinical Infectious Diseases 2012, 54 (6), 755-771; Strom, R. G.; Pacione, D.; Kalhorn, S. P.; Frempong-Boadu, A. K., Decreased risk of wound infection after posterior cervical fusion with routine local application of vancomycin powder. Spine (Phila Pa 1976) 2013, 38 (12), 991-4; Wukich, D. K.; Dikis, J. W.; Monaco, S. J.; Strannigan, K.; Suder, N. C.; Rosario, B. L., Topically Applied Vancomycin Powder Reduces the Rate of Surgical Site Infection in Diabetic Patients Undergoing Foot and Ankle Surgery. Foot Ankle Int 2015, 36 (9), 1017-24; Charlton, C. L., Hindler, J. A., Turnidge, J., & Humphries, R. M. Precision of vancomycin and daptomycin MICs for methicillin-resistant Staphylococcus aureus and effect of subculture and storage. Journal Clin Micro. 2014, 52 (11), 3898-3905).

The combination of commonly utilized surgical aids, gelatin as a hemostatic agent, and vancomycin as a highly effective antibiotic against MRSA, yields an efficient and safe method for delivering both to aid in the prevention of SSIs.

The following non-limiting examples illustrate exemplary systems and components thereof in accordance with various embodiments of the disclosure. The examples are merely illustrative and are not intended to limit the disclosure in any way. While the examples are drawn to antibiotics, chemotherapeutics, and NSAIDs, other agents are contemplated as being useful for incorporation in the hemostatic agent of the invention described herein.

### Example 1—Vancomycin and Daptomycin in Gelatin

The antibiotic-eluting hemostatic agent described herein provides for the continuous release of antibiotics for a minimum of 3 weeks, which exceeds the release previously shown for vancomycin. (Shukla, A.; Fang, J. C.; Puranam, S.; Hammond, P. T., Release of vancomycin from multilayer coated absorbent gelatin sponges. J Control Release 2012, 157 (1), 64-71; Zhou, J.; Fang, T.; Wang, Y.; Dong, J., The controlled release of vancomycin in gelatin/β-TCP composite scaffolds. Journal of biomedical materials research Part A 2012, 100 (9), 2295-2301; Shukla, A.; Avadhany, S. N.; Fang, J. C.; Hammond, P. T., Tunable Vancomycin Releasing Surfaces for Biomedical Applications. Small 2010, 6 (21), 2392-04).

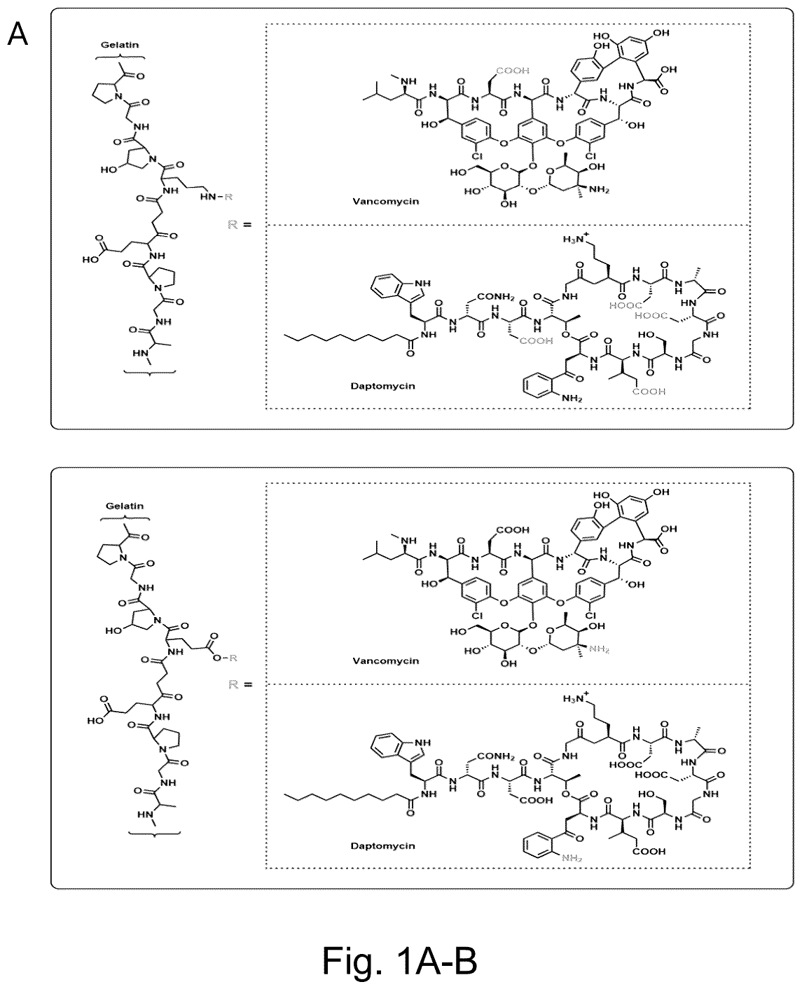

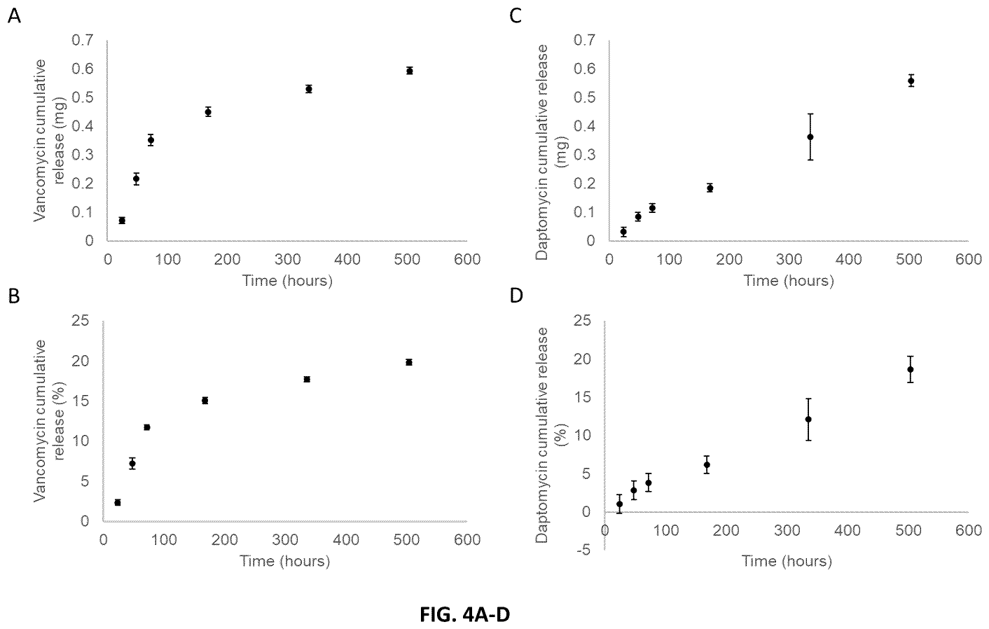

This functionality is accomplished through the use of carbodiimide chemistry to create crosslinked gelatin cages to entrap the antibiotic for immediate release while allowing for direct antibiotic conjugation with gelatin, providing a delayed release. As shown in FIG. 1, the degree of crosslinking between the gelatin and the antibiotic is associated with the availability of carboxyl and amino functional groups on the antibiotic. Hence, more available groups lead to additional conjugation (formation of amide bonds) between the gelatin and antibiotic. Increased rates of amide bond formation between the antibiotic and gelatin lead to slower release as depicted within the comparison of vancomycin and daptomycin shown in FIG. 4, as daptomycin has three additional carboxylates that can be used for conjugation.

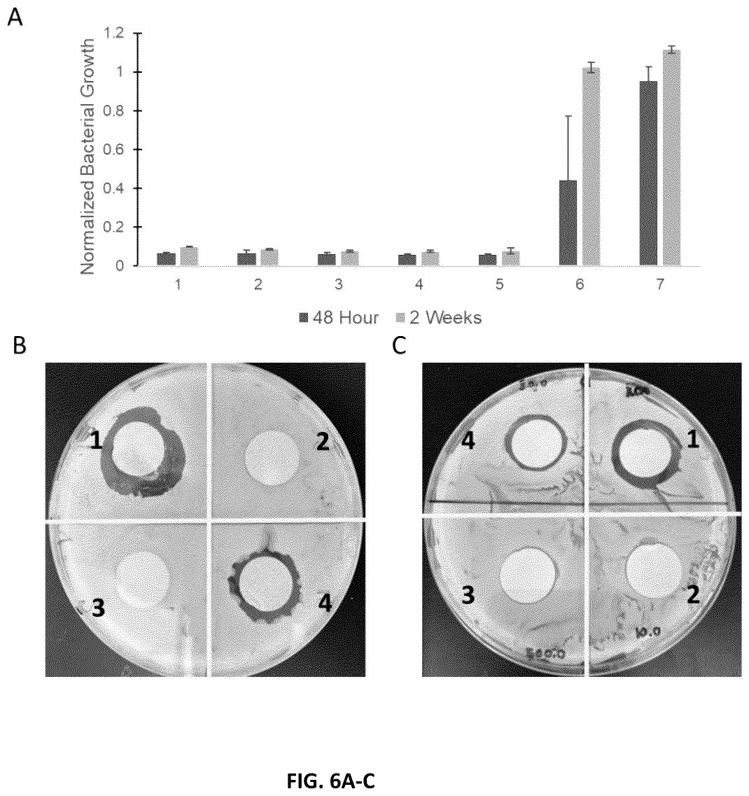

Analysis to determine concentrations of antibiotic released from the hemostatic agent via HPLC revealed that the structure of the released antibiotic differed slightly in later release samples. Retention times of the samples following the 24-hour release were slightly longer than those of the standards. This was likely due to dimerization or trimerization of the antibiotic, antibiotic conjugation to short amino acid sequences derived from gelatin, or a combination of the two. Microdilution and modified Kirby-Bauer assays shown in FIG. 6 reveal that vancomycin released from the hemostatic agent maintains its ability to inhibit the growth of S. aureus after a 2-week period. Taken together, these experiments indicate that even though the structure of the antibiotic may have changed slightly in later release samples, the potentially modified, released vancomycin maintains efficacy.

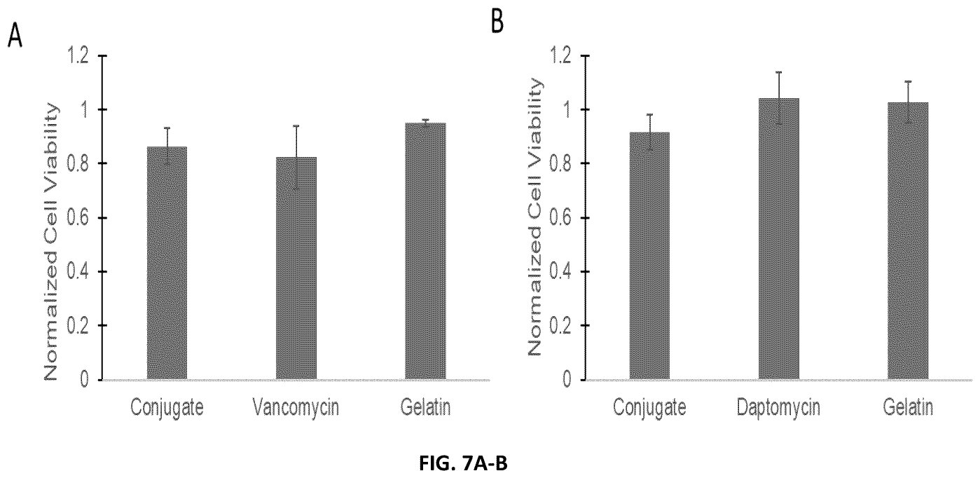

The antibiotic-eluting hemostatic agents developed here incorporate a crosslinked gelatin with an antibiotic. In some embodiments the crosslinker can be EDC. Although EDC itself is highly toxic, it has been shown to be minimally toxic following complete reactivity with its preferred functional groups. (Zhou, J.; Fang, T.; Wang, Y.; Dong, J., The controlled release of vancomycin in gelatin/β-TCP composite scaffolds. Journal of biomedical materials research Part A 2012, 100 (9), 2295-2301; Cammarata, C. R.; Hughes, M. E.; Ofner, C. M., 3rd, Carbodiimide induced crosslinking, ligand addition, and degradation in gelatin. Mol Pharm 2015, 12 (3), 783-93). The method in the current study utilized differences in solubility to minimize free or unreacted EDC in the final product, thereby ensuring that the antibiotic-eluting hemostatic agent is biocompatible as shown in FIG. 7. Additionally, studies in which gelatin was complexed with beta-TCP via various concentrations of EDC, showed that minimal concentrations of EDC (up to 10 mg/mL) were negligibly cytotoxic to cultured cells. (Zhou, J.; Fang, T.; Wang, Y.; Dong, J., The controlled release of vancomycin in gelatin/β-TCP composite scaffolds. Journal of biomedical materials research Part A 2012, 100 (9), 2295-2301).

### Materials and Methods

### Materials

Gelatin type B, 2-(N-morpholino) ethanesulfonic acid (MES), vancomycin, daptomycin, ceftazidime, 1-ethyl-3-(3-dimethylaminopropyl) carbodiimide (EDC), and absolute ethanol were purchased from Fischer Scientific (Waltham, MA). Ceftibuten and NaCl were purchased from Sigma-Aldrich (St. Louis, MO). Tryptic soy agar and broth were purchased from Difco Laboratories (Sparks, MD). HEK-293 fibroblasts and Staphylococcus aureus 25923 were obtained from ATCC (Manassas, VA).

### Preparation of Antibiotic-Eluting Hemostatic Agents

Gelatin was prepared as a 20 mg/mL stock solution in 0.05 M MES buffer pH 5.0 with stirring at 50° C. until soluble. To prepare the hemostatic agents, 20 mM carboxyl group concentration from the gelatin stock was incubated with either vancomycin (2.07 mM), or daptomycin (1.85 mM), under activation by EDC (60 mM) for 2 hours at 50 rpm and 22° C. The product was precipitated under ice-cold absolute ethanol followed by centrifugation at 6000×g, then dissolved in 1.85 mM NaCl for washing, followed by a second round of precipitation. Hemostatic agents were vacuum dried and heated at 80° C. to remove residual ethanol. The hemostatic agents are in a clumpy powder form after heating. The hemostatic agents may be administered in powder form or alternatively, an amount of phosphate buffered saline (PBS) may be added to achieve a paste-like consistency.

Varying the concentration of crosslinking agent and the reaction time highly impact the structure of the hemostatic agent. In some embodiments, the concentration of EDC is between about 20 mM and about 60 mM. Reaction time can be between about 1 hour to about 24 hours. In some embodiments, 0.1 M NaHCO may be used in place of 0.05 M MES buffer.

### Structural Analysis of Hemostatic Agents

Hemostatic agent morphology was examined by scanning electron microscopy (SEM) using a JEOL JSM-6490 microscope. Hemostatic agents were fixed in 10% formaldehyde for 2 hours, dehydrated with ethanol, and selected for mounting on double-sided conductive carbon tape.

Antibiotic Release from Antibiotic-Eluting Hemostatic Agents

For release profile determination, hemostatic agents were combined with 0.5 mL phosphate-buffered saline solution (PBS) to make a semisolid followed by immersion in 1 mL PBS. Samples were incubated at 37° C. and 100% relative humidity over a period of 3 weeks with 1 mL of PBS removed and then replaced with fresh PBS at 24, 48, 72, 96, 168, 336, and 504 hours. Release samples were stored at −80° C. until analysis to determine drug release kinetics of the hemostatic agents. Release samples were filtered through syringe filters (0.45 μm) and were analyzed by high performance liquid chromatography (Waters HPLC, 1100 series) to determine antibiotic concentrations. Samples were run for 10 min using 70/30 PBS/methanol mobile phase, 1 mL/min flow rate with a 150 μL injection volume on a C18 reverse phase column (Supelco) coupled with UV detection (280 nm for vancomycin or 223 nm for daptomycin). Peak height was correlated with standards of known concentrations of the perspective antibiotic used in the hemostatic agent to determine antibiotic concentration in the released samples.

### Fourier Transform Infrared Spectroscopy

FTIR spectra were collected on a Nicolet Is10 to ensure structural integrity of antibiotics released from the hemostatic agents. Released samples were scanned in the range between 4000 and 800 cm⁻¹.

### Bacterial Growth Inhibition and Efficacy of Released Antibiotic

Inhibition of S. aureus 25293 or E. Coli (Migula) Castellani and Chalmers by antibiotic-eluting hemostatic agents was determined through modified Kirby-Bauer disk diffusion tests and microdilution assays. Throughout all Kirby-Bauer tests, agar plates were formed using tryptic soy agar. The plates were subsequently coated with the appropriate strain within its exponential growth phase at a concentration of 10⁸ CFU/mL. Filter papers that had been incubated with the hemostatic agents, gelatin, or pure antibiotic equivalent to 24-hour release concentrations for two hours were then immediately placed upon the plates. Zones of inhibition were measured and photographed following 16-18 hours of incubation at 37° C. Microdilution assays were performed in triplicate using the appropriate bacteria in 96-well plates and done in serial two-fold dilutions. Bacteria grown to its exponential growth phase diluted in tryptic soy broth to 10⁵ CFU/mL was used for the assays. Following 16-18-hour incubation at 37° C., optical density at 600 nm for treated and control bacteria was examined via a BioTek PowerWave XS plate reader. The normalized bacterial inhibition was then calculated from Equation (1).

$$Normalized bacteria inhibition = \frac{( OD 600 positive control - OD 600 sample )}{( OD 600 positive control - OD 600 negative control )} \qquad ( 1 )$$

### Biocompatibility Analysis of Antibiotic-Eluting Hemostatic Agents

Hemostatic agent biocompatibility was established by examining cell viability of HEK-293T fibroblasts upon exposure to hemostatic agent eluent, pure antibiotic, and crosslinked gelatin samples. Cells were maintained in complete media (DMEM supplemented with 10% fetal bovine serum, 1 mM sodium pyruvate, and 1 mM penicillin/streptomycin at 37° C. in 5% CO₂). For analysis, cells were seeded at 10,000 cells per well in 96 well polystyrene tissue culture plates and incubated in complete media (100 μL) at 37° C. for 24 hours. Hemostatic agents were allowed to completely elute in culture media at 37° C. for 24 hours. Gelatin (0.3 mg/mL) and pure antibiotics (0.1 mg/mL) were also incubated in culture media for 24 hours. All samples were filtered through 0.2 μm filters, and the media on the growing cells was replaced with of test media (100 μL). Positive controls were cells cultured in untreated media, while negative controls contained untreated media and no cells. Following 18-hour exposure to the test media, dye solution (15 μl) was administered to each well and the plate was incubated at 37° C. for 3 hours in a humidified, 5% CO₂ atmosphere. After incubation, solubilization solution/stop mix (100 μl) was added to each well. A BioTek PowerWave XS plate reader was used to detect the absorbance of the wells at 600 nm to elucidate cell viability. Cell viability was calculated from Equation (2).

$$Normalized cell viability = \frac{( Abs 600 sample - Abs 600 negative control )}{( Abs 600 positive control - Abs 600 negative control )} \qquad ( 2 )$$

### Results

### Characterization of Antibiotic-Eluting Hemostatic Agents

Gelatin type-B was used for all conjugations as it is known to possess increased concentrations of carboxylate groups in relation to other forms. Slightly acidic conditions (pH 5-7) in 0.05 M MES ensured the availability of carboxylate groups for nucleophilic attack by EDC to promote enhanced reactivity in an effort to generate highly crosslinked gelatin. (Kosasih, A. B., B. J.; Wigent, R. J.; & Ofner III, C. M., Characterization and in vitro release of methotrexate from gelatin/methotrexate conjugates formed using different preparation variables. International Journal of Pharmaceutics 2000, 204 (1), 81-89). Crosslinking reactions performed in the presence of antibiotics allowed for the trapping of antibiotics in cage-like, crosslinked gelatin structures in addition to the direct conjugation of the antibiotic to the gelatin. Possible binding outcomes of vancomycin or daptomycin with gelatin are illustrated in FIG. 1.

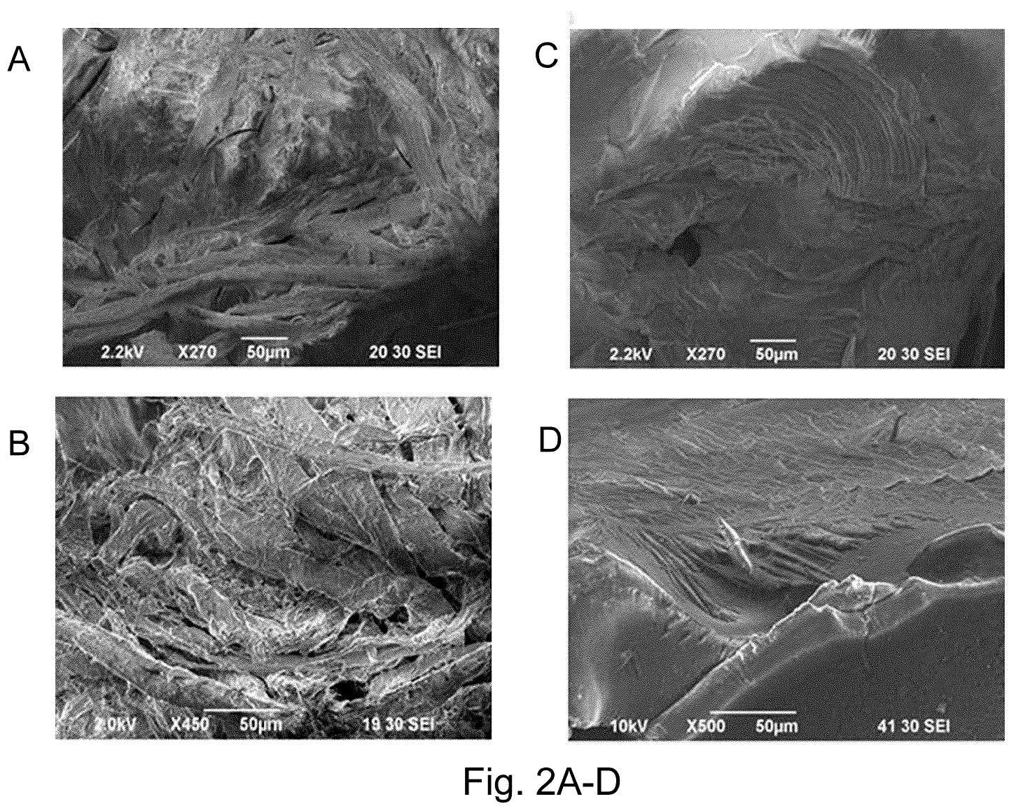

SEM micrographs of the prepared hemostatic agents containing either vancomycin or daptomycin are shown in FIG. 2. Upon examination of the surface features of the hemostatic agents, a compact glassy layered morphology was seen. There were slight differences between the vancomycin and daptomycin-linked hemostatic agents as the vancomycin hemostatic agents demonstrated increased surface variations and “pitting” (FIG. 2B) in comparison to the smoother morphology of the daptomycin hemostatic agents (FIG. 2D). This is likely due to the increased availability of potential binding groups in the structure of daptomycin, which is likely to lead to increased rates of daptomycin-gelatin conjugation and decreased rates of gelatin-gelatin conjugation.

### Swelling Capacity of Antibiotic-Eluting Hemostatic Agents

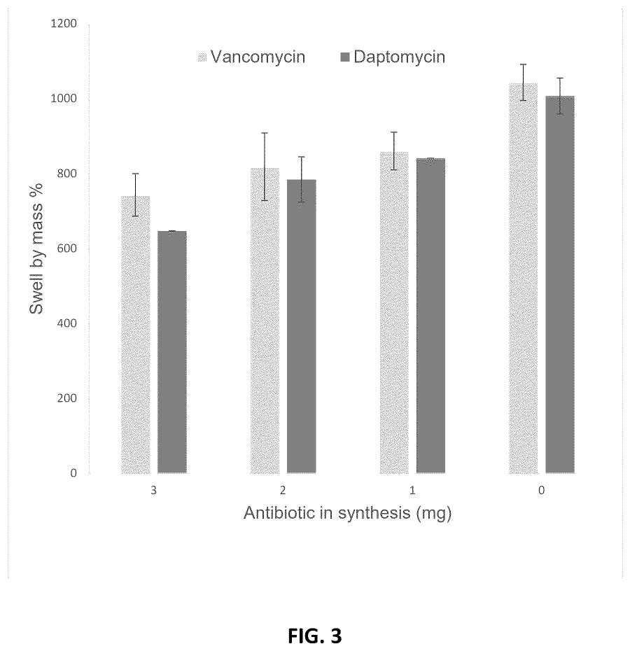

The swelling behavior of a hemostatic agent plays a significant role in absorption of body fluids, metabolites, and regulating nutrients. FIG. 3 shows the swelling behavior of the antibiotic-eluting hemostatic agents with varying concentrations of antibiotic used for each. Compared with conjugated gelatin alone, addition of either vancomycin or daptomycin slightly decreased the swelling capacity of the gelatin. Both vancomycin and daptomycin conjugates exhibited slightly decreased swelling with higher concentrations of antibiotic with a more notable decrease with the daptomycin. This difference can likely be attributed to the additional binding sites on daptomycin which may allow for additional bond formation between the daptomycin and gelatin leading to a more rigid complex. While there was a decrease in swelling of the hemostatic agents, each possessed the ability to swell to more than 600% of their original size and as such, can serve as effective hemostats.

Antibiotic Release from Hemostatic Agents

Release profiles of vancomycin or daptomycin-linked hemostatic agents determined by HPLC are shown in FIG. 4. Vancomycin hemostatic agents revealed a large initial burst effect with a release of 10% of the loaded vancomycin within 48 hours as seen in FIG. 4B. Following the initial burst, release of vancomycin was sustained for over a period of 500 hours until separation was no longer practical.

Daptomycin hemostatic agents initially showed a much slower, linear release likely due to increased rates of direct binding to gelatin and less free daptomycin trapped within the crosslinked gelatin (FIGS. 4A and 4C). Akin to the vancomycin hemostatic agents, the daptomycin hemostatic agents released approximately 20% of the total antibiotic near 500 hours and the complex slowly collapsed following that period (FIG. 4D).

Structure and Efficacy of Released Antibiotics from Hemostatic Agents

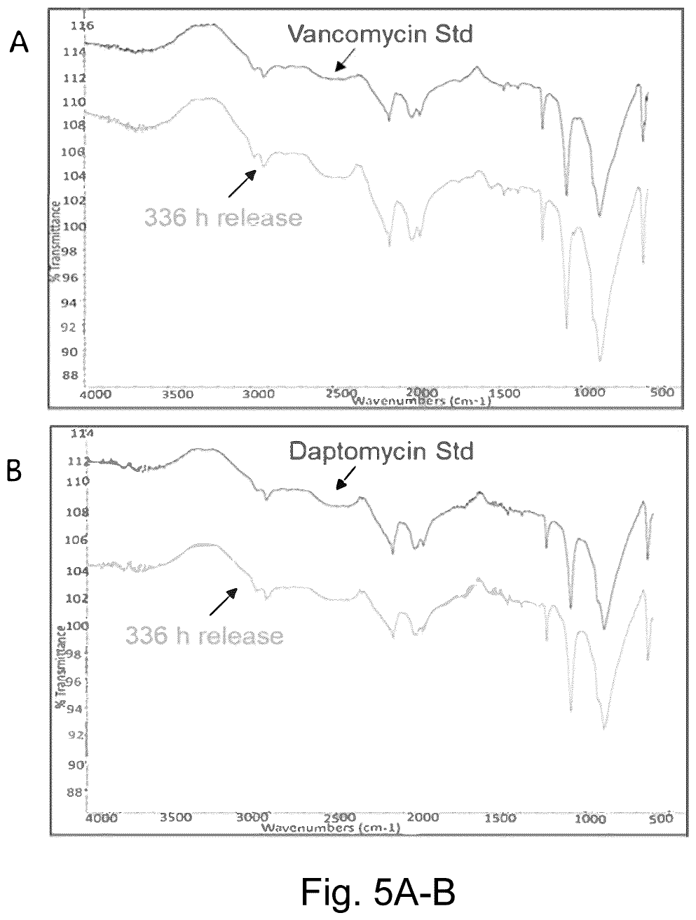

Conjugation of antibiotics to gelatin via the activity of EDC may cause structural changes to the antibiotic itself. As such, FTIR spectral data was obtained of the released antibiotic samples. Basic chemical stability of vancomycin and daptomycin were confirmed in all released samples as seen in the representative samples depicted in FIG. 5.

To determine the efficacy of vancomycin released from antibiotic-eluting hemostatic agents, microdilution assays were performed. Therapeutic efficiency was established as shown in FIG. 6A through monitoring the normalized density of S. aureus cultured within the presence of dilutions of released vancomycin. Efficacy remained consistent between 48-hour and 336-hour samples, further indicating structural integrity of the released vancomycin and its retained ability to sustain inhibition of bacterial growth over the course of the release. Vancomycin released from the hemostatic agent displayed an MIC between 0.5 and 2 μg/mL as previously shown in the literature for pure vancomycin. (Kshetry, A. O.; Pant, N. D.; Bhandari, R.; Khatri, S.; Shrestha, K. L.; Upadhaya, S. K.; Poudel, A.; Lekhak, B.; Raghubanshi, B. R., Minimum inhibitory concentration of vancomycin to methicillin resistant Staphylococcus aureus isolated from different clinical samples at a tertiary care hospital in Nepal. Antimicrobial Resistance & Infection Control 2016, 5 (1), 27; Van Hal, S.; Lodisc, T. P.; Paterson, D. L., The clinical significance of vancomycin minimum inhibitory concentration in Staphylococcus aureus infections: a systematic review and meta-analysis. Clinical Infectious Diseases 2012, 54 (6), 755-771; Strom, R. G.; Pacione, D.; Kalhorn, S. P.; Frempong-Boadu, A. K., Decreased risk of wound infection after posterior cervical fusion with routine local application of vancomycin powder. Spine (Phila Pa 1976) 2013, 38 (12), 991-4; Wukich, D. K.; Dikis, J. W.; Monaco, S. J.; Strannigan, K.; Suder, N. C.; Rosario, B. L., Topically Applied Vancomycin Powder Reduces the Rate of Surgical Site Infection in Diabetic Patients Undergoing Foot and Ankle Surgery. Foot Ankle Int 2015, 36 (9), 1017-24).

Modified Kirby-Bauer Assays further confirmed the activity of vancomycin released from hemostatic agents as seen in FIG. 6B. Filter paper with dried antibiotic-eluting hemostatic agent, pure vancomycin, crosslinked gelatin, or filter paper only was placed on S. aureus coated agar. A clear zone of inhibition (ZOI) surrounds the vancomycin control (FIG. 6B, panel 4) as well as the antibiotic-eluting hemostatic agent (FIG. 6B, panel 1) which indicated that there was inhibition of S. aureus growth.

Similarly, modified Kirby-Bauer Assays also further confirmed the activity of daptomycin released from hemostatic agents as seen in FIG. 6C. Filter paper with dried antibiotic-eluting hemostatic agent, pure daptomycin, crosslinked gelatin, or filter paper only was placed on S. aureus coated agar. A clear zone of inhibition (ZOI) surrounds the daptomycin control (FIG. 6C, panel 4) as well as the antibiotic-eluting hemostatic agent (FIG. 6C, panel 1) which indicated that there was inhibition of S. aureus growth.

Biocompatibility of Antibiotics Released from Hemostatic Agents

While vancomycin and gelatin have both separately been shown to be minimally toxic to mammalian cells, combination of the two components in the presence of EDC has not. (Shukla, A.; Avadhany, S. N.; Fang, J. C.; Hammond, P. T., Tunable Vancomycin Releasing Surfaces for Biomedical Applications. Small 2010, 6 (21), 2392-04; Yang, G.; Xiao, Z.; Long, H.; Ma, K.; Zhang, J.; Ren, X.; Zhang, J., Assessment of the characteristics and biocompatibility of gelatin sponge scaffolds prepared by various crosslinking methods. Sci Rep. 2018, 8 (1), 1616). Therefore, cell viability of human fibroblasts in the presence of the antibiotic-eluting hemostatic agents was assessed. Media incubated with prepared hemostatic agents containing released antibiotic was applied to cultured cells, as well as control concentrations of antibiotic, and media incubated with crosslinked gelatin. As shown in FIG. 7, cell viability of all released samples was comparable to untreated healthy cells illustrating that the antibiotic released from the hemostatic agent is nontoxic to mammalian cells.

### Example 2—Cephalosporins and Quinolones in Gelatin

The antibiotic-eluting hemostatic agent described herein provides for the continuous release of antibiotics for a minimum of 3 weeks. As with Example 1, described above, this functionality is accomplished through the use of carbodiimide chemistry to create crosslinked gelatin cages to entrap the antibiotic for immediate release while allowing for direct antibiotic conjugation with gelatin, providing a delayed release.

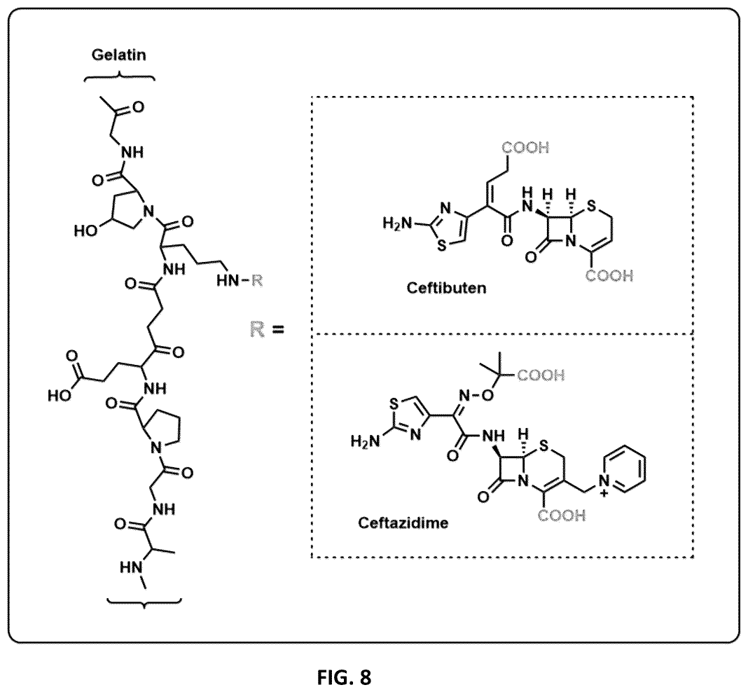

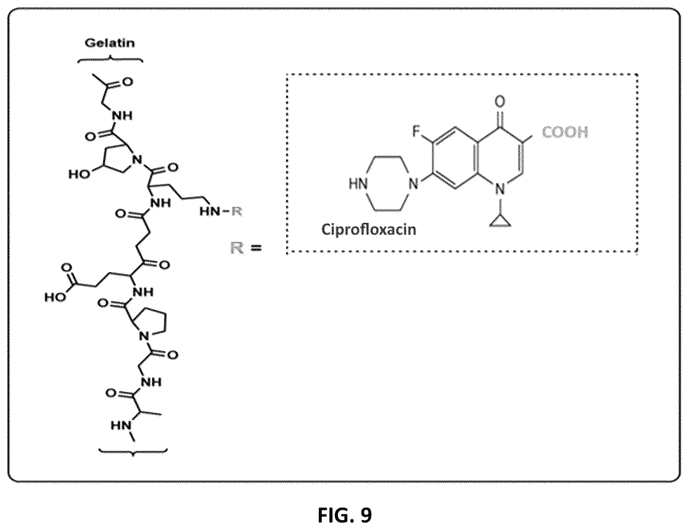

As shown in FIGS. 8 and 9, the degree of crosslinking between the gelatin and the antibiotic is associated with the availability of carboxyl and amino functional groups on the antibiotic. Hence, more available groups lead to additional conjugation (formation of amide bonds) between the gelatin and antibiotic. Increased rates of amide bond formation between the antibiotic and gelatin lead to slower release.

### Materials and Methods

### Materials

Gelatin type B, 2-(N-morpholino) ethanesulfonic acid (MES), ceftazidime, ceftibuten, ciprofloxacin, 1-ethyl-3-(3-dimethylaminopropyl) carbodiimide (EDC), and absolute ethanol were purchased from Fischer Scientific (Waltham, MA). Ceftibuten and NaCl were purchased from Sigma-Aldrich (St. Louis, MO). Tryptic soy agar and broth were purchased from Difco Laboratories (Sparks, MD). HEK-293T fibroblasts and E. Coli (Migula) Castellani and Chalmers were obtained from ATCC (Manassas, VA).

### Preparation of Antibiotic-Eluting Hemostatic Agents

Gelatin was prepared as a 20 mg/mL stock solution in 0.05 M MES buffer pH 5.0 with stirring at 50° C. until soluble. To prepare the hemostatic agents, 20 mM carboxyl group concentration from the gelatin stock was incubated with either ceftibuten (7.31 mM) or ceftazidime (5.49 mM) or ciprofloxacin (0.19 mM) under activation by EDC (60 mM) for 2 hours at 50 rpm and 22° C. The product was precipitated under ice-cold absolute ethanol followed by centrifugation at 6000×g, then dissolved in 1.85 mM NaCl for washing, followed by a second round of precipitation. Hemostatic agents were vacuum dried and heated at 80° C. to remove residual ethanol.

### Structural Analysis of Hemostatic Agents

Hemostatic agent morphology was examined by scanning electron microscopy (SEM) using a JEOL JSM-6490 microscope. Hemostatic agents were fixed in 10% formaldehyde for 2 hours, dehydrated with ethanol, and selected for mounting on double-sided conductive carbon tape.

### Degree of Swelling of Hemostatic Agents

Samples were prepared as above and complete vacuum dried samples were weighed and measured then immersed in PBS at 37° C. in a humidity-controlled chamber for 24 hours. The sample was removed, blot-dried with an absorbent paper to remove the excess solution and weighed. Swelling capacity was calculated by subtracting the initial mass of the sample from the final mass of the sample after 24 hours as a percentage increase.

Antibiotic Release from Antibiotic-Eluting Hemostatic Agents

For release profile determination, hemostatic agents were combined with 0.5 mL phosphate-buffered saline solution (PBS) to make a semisolid followed by immersion in 1 mL PBS. Samples were incubated at 37° C. and 100% relative humidity over a period of 3 weeks with 1 mL of PBS removed and then replaced with fresh PBS at 24, 48, 72, 96, 168, 336, and 504 hours. Release samples were stored at −80° C. until analysis to determine drug release kinetics of the hemostatic agents. Release samples were filtered through syringe filters (0.45 μm) and were analyzed by high performance liquid chromatography (Waters HPLC, 1100 series) to determine antibiotic concentrations. Samples were run for 10 min using 70/30 PBS/methanol mobile phase, 1 mL/min flow rate with a 150 μL injection volume on a C18 reverse phase column (Supelco) coupled with UV detection (254 for ceftazidime, 228 for ceftibuten, or 277 for ciprofloxacin). Peak height was correlated with standards of known concentrations of the perspective antibiotic used in the hemostatic agent to determine antibiotic concentration in the released samples.

### Fourier Transform Infrared Spectroscopy

FTIR spectra were collected on a Nicolet Is10 to ensure structural integrity of antibiotics released from the hemostatic agents. Released samples were scanned in the range between 4000 and 800 cm⁻¹.

### Biocompatibility Analysis of Antibiotic-Eluting Hemostatic Agents

Hemostatic agent biocompatibility was established by examining cell viability of HEK-293T fibroblasts upon exposure to hemostatic agent eluent, pure antibiotic, and crosslinked gelatin samples. Cells were maintained in complete media (DMEM supplemented with 10% fetal bovine serum, 1 mM sodium pyruvate, and 1 mM penicillin/streptomycin at 37° C. in 5% CO₂). For analysis, cells were seeded at 10,000 cells per well in 96 well polystyrene tissue culture plates and incubated in complete media (100 μL) at 37° C. for 24 hours. Hemostatic agents were allowed to completely elute in culture media at 37° C. for 24 hours. Gelatin (0.3 mg/mL) and pure antibiotics (0.1 mg/mL) were also incubated in culture media for 24 hours. All samples were filtered through 0.2 μm filters, and the media on the growing cells was replaced with of test media (100 μL). Positive controls were cells cultured in untreated media, while negative controls contained untreated media and no cells. Following 18-hour exposure to the test media, dye solution (15 μl) was administered to each well and the plate was incubated at 37° C. for 3 hours in a humidified, 5% CO₂ atmosphere. After incubation, solubilization solution/stop mix (100 μl) was added to each well. A BioTek PowerWave XS plate reader was used to detect the absorbance of the wells at 600 nm to elucidate cell viability. Cell viability was calculated from Equation (2).

$$Normalized cell viability = \frac{( Abs 600 sample - Abs 600 negative control )}{( Abs 600 positive control - Abs 600 negative control )} \qquad ( 2 )$$

### Results

### Characterization of Antibiotic-Eluting Hemostatic Agents

Crosslinking reactions performed in the presence of antibiotics allowed for the trapping of antibiotics in cage-like, crosslinked gelatin structures in addition to the direct conjugation of the antibiotic to the gelatin. Possible binding outcomes of ceftazidime or ceftibuten with gelatin are illustrated in FIG. 8. Possible binding outcome of ciprofloxacin is shown in FIG. 9.

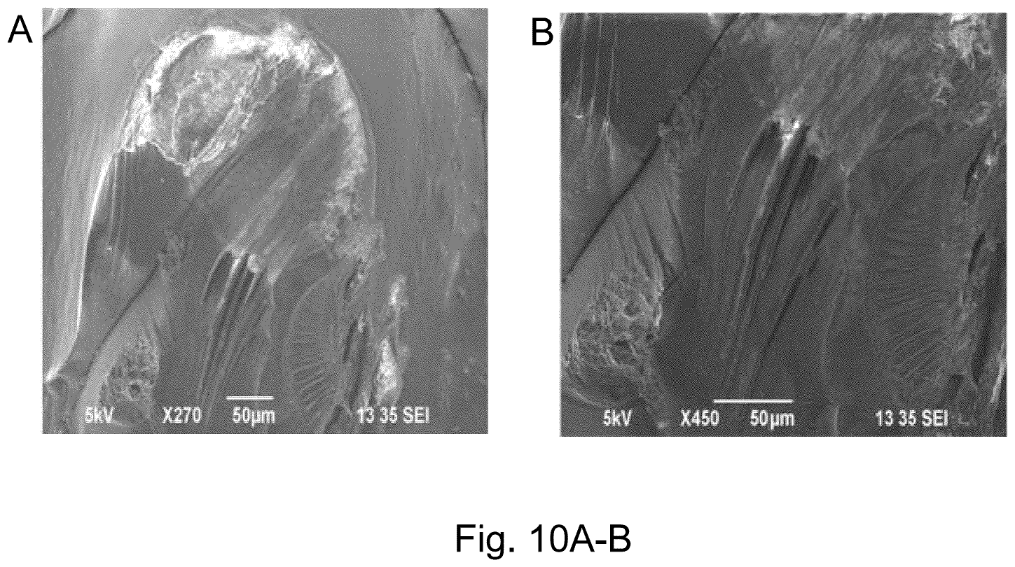

SEM micrographs of the prepared hemostatic agents containing ceftazidime are shown in FIG. 10. Upon examination of the surface features of the hemostatic agents, a compact glassy layered morphology was seen. Ceftazidime hemostatic agents demonstrate a smoother morphology similar to that of the daptomycin hemostatic agents (FIG. 2). This is likely due to the increased reactivity of smaller antibiotics such as ciprofloxacin, ceftazidime, and ceftibuten. Reactions including these smaller structures were exothermic and literally hot to the touch when combined with EDC and gelatin. The smaller structure may yield increased rates of cephalosporin-gelatin conjugation and decreased rates of gelatin-gelatin conjugation.

### Swelling Capacity of Antibiotic-Eluting Hemostatic Agents

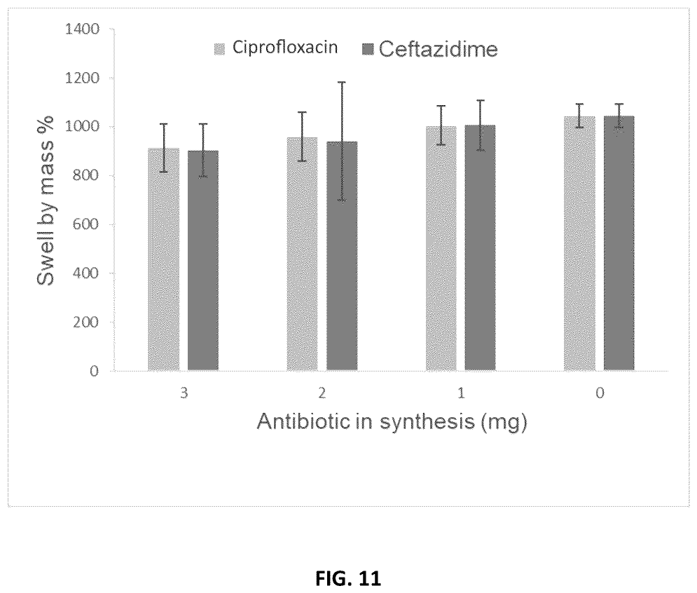

The swelling behavior of a hemostatic agent plays a significant role in absorption of body fluids, metabolites, and regulating nutrients. FIG. 11 shows the swelling behavior of the antibiotic-eluting hemostatic agents with varying concentrations of antibiotic used for each. Compared with conjugated gelatin alone, both ciprofloxacin and ceftazidime conjugates had similar swelling profiles and swelling was decreased slightly by higher concentrations of antibiotic.

Antibiotic Release from Hemostatic Agents

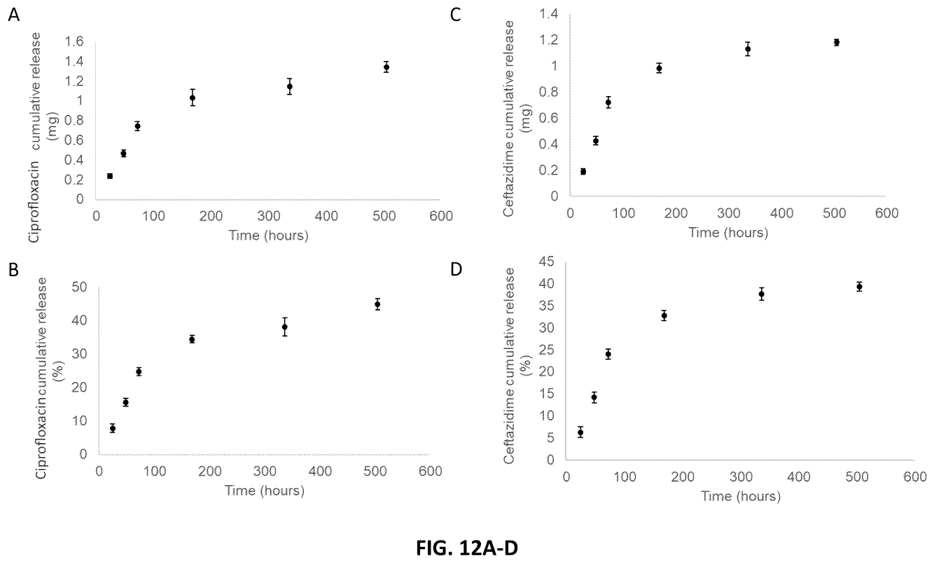

Release profiles of ceftazidime-linked and ciprofloxacin-linked hemostatic agents determined by HPLC is shown in FIG. 12A-D. Ceftazidime and ciprofloxacin hemostatic agents showed steady and nearly equal release at earlier collection points up to 1 week. Following 1 week, slower release of ceftazidime and ciprofloxacin was sustained for over a period of 500 hours until separation was no longer practical.

Structure and Efficacy of Released Antibiotics from Hemostatic Agents

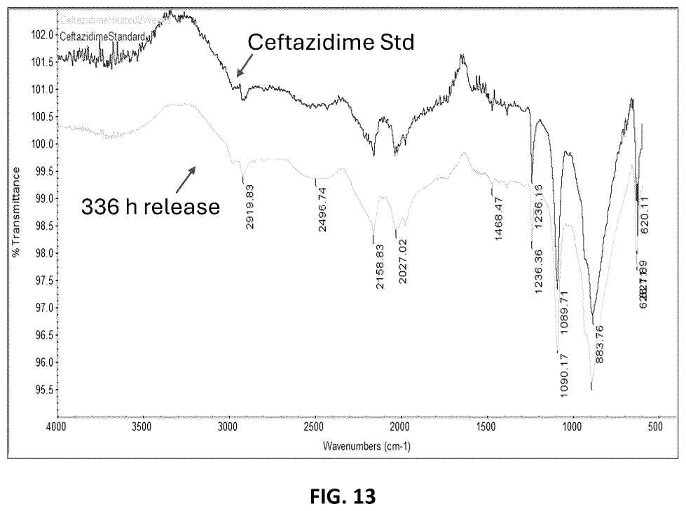

Conjugation of antibiotics to gelatin via the activity of EDC may cause structural changes to the antibiotic itself. As such, FTIR spectral data was obtained of the released antibiotic samples. Basic chemical stability of ceftazidime was confirmed in all released samples as seen in the representative samples depicted in FIG. 13.

Biocompatibility of Antibiotics Released from Hemostatic Agents

Cell viability of human fibroblasts in the presence of the antibiotic-eluting hemostatic agents was assessed. Media incubated with prepared hemostatic agents containing released antibiotic was applied to cultured cells, as well as control concentrations of antibiotic, and media incubated with crosslinked gelatin.

### Example 3—Non-Steroidal Anti-Inflammatory Drug (NSAID) Eluting Gelatin Hemostatic Agents

The NSAID-eluting hemostatic agent described herein provides for the continuous release of diclofenac for a minimum of 2 weeks. Similar to the antibiotic-eluting hemostatic agents described above, this functionality is accomplished through the use of carbodiimide chemistry to create crosslinked gelatin cages to entrap the NSAID for immediate and early delayed release while allowing for direct NSAID conjugation with gelatin, providing a sustained release.

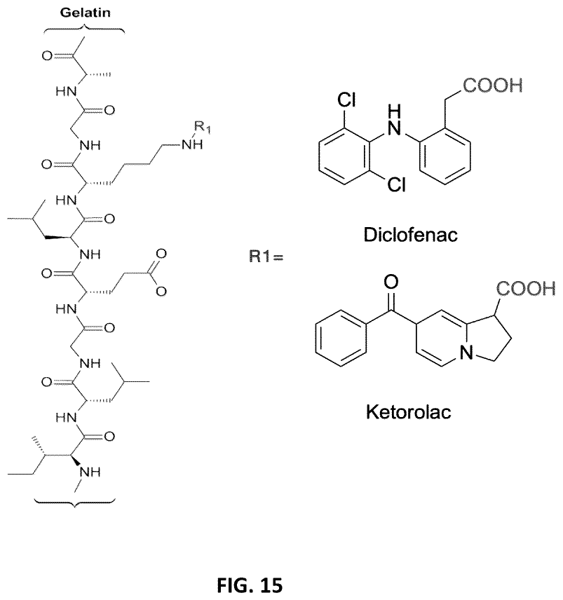

As shown in FIG. 15, the degree of crosslinking between the gelatin and the NSAID is associated with the availability of carboxyl and amino functional groups on the NSAID. Hence, more available groups lead to additional conjugation (formation of amide bonds) between the gelatin and NSAID. Increased rates of amide bond formation between the NSAID and gelatin lead to slower release.

### Materials and Methods

### Materials

Gelatin type B, Na₃PO₄, diclofenac, ketorolac, 1-ethyl-3-(3-dimethylaminopropyl) carbodiimide (EDC), and absolute ethanol were purchased from Fisher Scientific (Waltham, MA). NaCl was purchased from Sigma-Aldrich (St. Louis, MO). HEK-293T fibroblasts were obtained from ATCC (Manassas, VA).

### Preparation of Antibiotic-Eluting Hemostatic Agents

Gelatin was prepared as a 20 mg/mL stock solution in 0.1 M phosphate buffer at pH 7.4 with stirring at 50° C. until soluble. To prepare the conjugates, 20 mM carboxyl group concentration from the gelatin stock was incubated with diclofenac or ketorolac (1 mM) under activation by EDC (60 mM) for 2 hours at 50 rpm and 22° C. The product was precipitated under ice-cold absolute ethanol followed by centrifugation at 6000×g, then dissolved in 1.85 mM NaCl for washing, followed by a second round of precipitation. Conjugates were vacuum dried and heated at 80° C. to remove residual ethanol.

Antibiotic Release from Antibiotic-Eluting Hemostatic Agents

For release profile determination, conjugates were combined with 0.5 mL phosphate-buffered saline solution (PBS) to make a semisolid followed by immersion in 1 mL PBS. Samples were incubated at 37° C. and 100% relative humidity over a period of 2 weeks with 1 mL of PBS removed and then replaced with fresh PBS at 24, 48, 120, 168, and 336 hours. Release samples were stored at −80° C. until analysis to determine drug release kinetics of the conjugates. Release samples were filtered through syringe filters (0.45 μm) and were analyzed by high performance liquid chromatography (Waters HPLC, 1100 series) to determine antibiotic concentrations. Samples were run for 10 min using 70/30 PBS/methanol mobile phase, 1 mL/min flow rate with a 150 μL injection volume on a C18 reverse phase column (Supelco) coupled with UV detection (276 for diclofenac). Peak height was correlated with standards of known concentrations of the perspective antibiotic used in the conjugate to determine antibiotic concentration in the released samples.

### Fourier Transform Infrared Spectroscopy

Fourier Transform Infrared Spectroscopy (FTIR) was performed to confirm the structural integrity of ketorolac released from the hemostatic conjugates. Spectral analysis was conducted using a Nicolet iS10 FTIR spectrometer. Eluted samples were prepared by incubating ketorolac-loaded conjugates in phosphate-buffered saline at 37° C. for 24 hours. Each sample was scanned in triplicate to ensure reproducibility.

### Biocompatibility Analysis of NSAID-Eluting Hemostatic Agents

To assess the biocompatibility of NSAID-eluting hemostatic agents, viability of HEK-293T fibroblast cells following exposure to eluent derived from cross-linked gelatin conjugates incorporating diclofenac or ketorolac was assessed. For comparative purposes, eluents from conjugates with no drug, pure NSAIDs, and gelatin-only samples were also examined. HEK-293T cells were cultured in complete Dulbecco's Modified Eagle Medium (DMEM) supplemented with 10% fetal bovine serum, 1 mM sodium pyruvate, and 1% penicillin-streptomycin at 37° C. in a humidified atmosphere with 5% CO₂. Cells were seeded at a density of 10,000 cells per well in 96-well polystyrene tissue culture plates and allowed to adhere for 24 hours in 100 μL of complete media. NSAID-eluting conjugates (diclofenac or ketorolac) were incubated in complete media at 37° C. for 48 hours to generate drug eluents. Control samples included gelatin-only matrices (0.3 mg/mL) and free diclofenac or ketorolac solutions (0.1 mg/mL), each prepared under identical incubation conditions. All samples were sterile filtered prior to cell exposure. Following media removal, each well received 100 μL of the corresponding test eluent. Positive controls consisted of untreated cells in fresh complete media, while negative controls contained media without cells. Following 18 hours of exposure to the test media, MTT assays were conducted, and absorbance was measured at 600 nm using a BioTek PowerWave XS plate reader. Cell viability was calculated relative to untreated controls to determine the cytocompatibility of each formulation.

Biocompatibility of NSAIDs Released from Hemostatic Agents

While diclofenac and ketorolac are widely used NSAIDs with established safety profiles, their incorporation into gelatin-based hemostatic matrices via EDC-mediated crosslinking warranted evaluation of their cytocompatibility upon release. To assess this, the viability of human fibroblast cells (HEK-293T) was measured following exposure to media incubated with NSAID-eluting hemostatic agents. Conditioned media was collected after 24-hour incubation of the prepared conjugates (diclofenac- or ketorolac-loaded) at 37° C. and applied to cultured HEK-293T cells. Control groups included cells treated with equivalent concentrations of free drug and media incubated with cross-linked gelatin lacking NSAIDs.

### Results

### Characterization of Antibiotic-Eluting Hemostatic Agents

Crosslinking reactions performed in the presence of diclofenac or ketorolac allowed for the trapping of the non-steroidal anti-inflammatory drug (NSAID) in cage-like, crosslinked gelatin structures in addition to the direct conjugation of the NSAID to the gelatin. Possible binding outcomes of diclofenac or ketorolac with gelatin are illustrated in FIG. 15.

### Hemostatic Agents

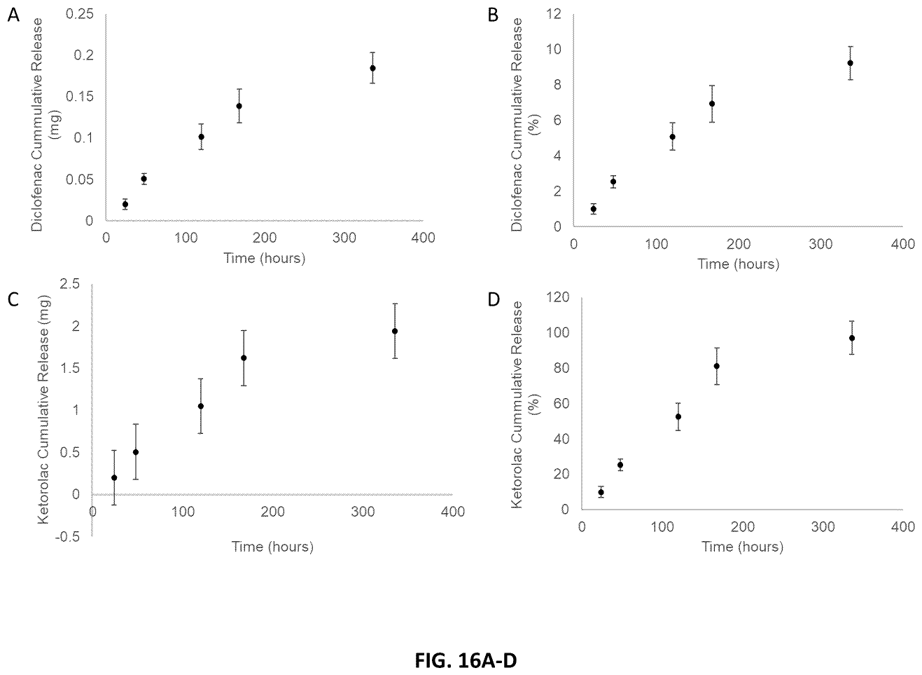

Release profiles of diclofenac or ketorolac eluting hemostatic agents were characterized using high-performance liquid chromatography (HPLC) as shown in FIG. 16. The two drug systems demonstrated distinct release behaviors likely due to their chemical structures and interactions with the gelatin matrix. Diclofenac-eluting conjugates displayed a steady and relatively uniform release pattern over a two-week period. The sustained release is likely due to diclofenac being physically sequestered within gelatin cages formed during the crosslinking process, as it lacks multiple functional groups to facilitate covalent bonding with the matrix. This entrapment may slow diffusion and enable a prolonged release profile suitable for sustained therapeutic effect.

Ketorolac-eluting conjugates, on the other hand, exhibited a more progressive release profile. Approximately 80% of the total ketorolac was released over the course of 200 hours with almost 100% being released over 2 weeks' time. The difference seen between diclofenac and ketorolac release may be attributed to steric strain around the carboxylate group of ketorolac, which could limit efficient conjugation directly to the gelatin or stable interaction with the gelatin network. Rather than being rapidly liberated, ketorolac appears to be more loosely associated with the matrix, allowing it to elute gradually over time.

Structure and Integrity of Released Ketorolac from Hemostatic Agents

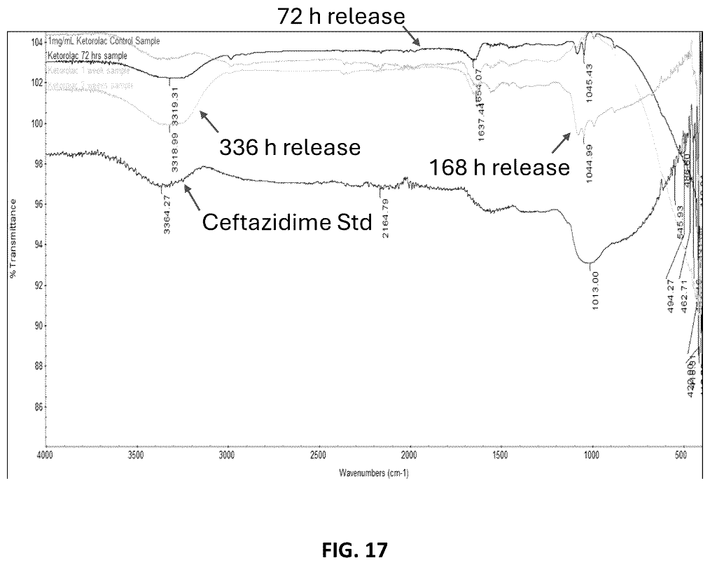

To assess whether conjugation to gelatin affected the chemical structure of ketorolac, FTIR spectroscopy was used to analyze the drug following its release from the hemostatic agents. Since conjugation reactions involving EDC may alter or degrade functional groups, spectral comparisons were conducted between released ketorolac and pure ketorolac standards as shown in FIG. 17.

Characteristic absorption peaks corresponding to functional groups in ketorolac were identified and compared to those of pure ketorolac reference spectra. The preservation of key functional group vibrations—such as carbonyl, aromatic, and hydroxyl stretches—indicated that the drug remained chemically intact following release from the conjugate matrix. These results, illustrated in representative spectra in FIG. 17, confirm the structural integrity of ketorolac throughout the conjugation and elution process, supporting its continued therapeutic efficacy post-release.

### Biocompatibility

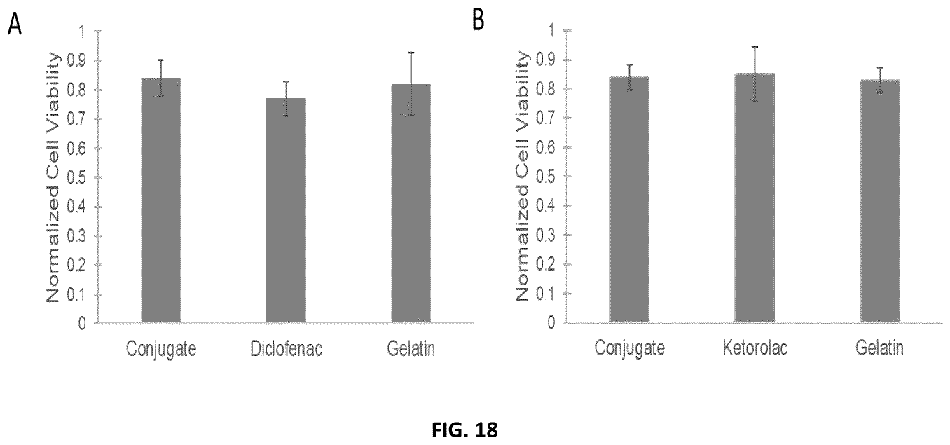

As shown in FIG. 18, cell viability across all treatment groups remained comparable to untreated controls. These results indicate that both diclofenac and ketorolac, when released from the gelatin-based hemostatic agents, maintain biocompatibility with mammalian cells. The absence of cytotoxic effects supports the potential clinical utility of these drug-eluting systems for localized anti-inflammatory delivery in hemostatic applications.

### Example 4—Gelatin Conjugates for Treatment of Cutaneous Squamous Cell Carcinoma

Cutaneous squamous cell carcinoma (cSCC) is a malignant skin tumor that poses a significant public health concern both in the United States and globally. Over 700,000 new cases of cSCC are diagnosed annually in the U.S., with incidence projected to rise by 50% by 2030. Although surgical excision remains the gold standard for treatment, it is not without limitations. Post-surgical recurrence occurs in approximately 8% of cases, prompting the need for adjunct therapies to further reduce recurrence rates and improve long-term outcomes. (Potenza, C.; Bernardini, N.; Balduzzi, V.; Losco, L.; Mambrin, A.; Marchesiello, A.; Tolino, E.; Zuber, S.; Skroza, N.; Proietti, I. A Review of the Literature of Surgical and Nonsurgical Treatments of Invasive Squamous Cells Carcinoma. BioMed Res. Int. 2018, 2018, 9489163).

Among these adjunctive options, Mohs micrographic surgery has demonstrated the highest efficacy in reducing recurrence, lowering the five-year rate to as low as 3%. However, this technique is time-consuming, costly, and often reserved for high-risk or cosmetically sensitive tumors. (Potenza, 2018). The inventors evaluated gelatin-based conjugates as an alternative treatment strategy to reduce cSCC recurrence. These systems are designed to release anticancer agents directly into post-surgical wound beds, potentially reducing recurrence while minimizing systemic exposure.

Gelatin is a low-cost, biocompatible, and biodegradable polymer with a long history of biomedical use. Its degradation products are nontoxic, and its high molecular weight allows for reduced renal clearance and prolonged circulation time. (A. Jiang, X.; Du, Z.; Zhang, X.; Zaman, F.; Song, Z.; Guan, Y.; Yu, T.; Huang, Y. Gelatin-Based Anticancer Drug Delivery Nanosystems: A Mini Review. Front. Bioeng. Biotechnol. 2023, 11, 1158749). These properties, combined with its ability to form and support sustained drug release, make gelatin a promising candidate for localized drug delivery systems. When placed at a surgical site, gelatin-based conjugates can act as scaffolds that gradually release anticancer agents into the surrounding tissue, mitigating recurrence through sustained therapeutic exposure.

The inventors examined gelatin conjugates incorporating three agents with known or potential efficacy against cSCC: 5-fluorouracil (5FU), doxorubicin, and curcumin. 5FU is a standard chemotherapeutic agent used topically and systemically for cSCC. (Wu, D. C.; Cammarata, C. R.; Park, H. J.; Rhodes, B. T.; Ofner, C. M. Preparation, Drug Release, AND Cell Growth Inhibition of a Gelatin—Doxorubicin Conjugate. Pharm. Res. 2013, 30 (8), 2087-2096; Ferriol, A.; Morán, M. del C. Enhanced Performance of Gelatin 5-Fluorouracil-Containing Nanoparticles against Squamous Cell Carcinoma in Simulated Chronic Wounds Conditions. Mater. Sci. Eng. C 2021, 124, 112073).

Curcumin, while not yet clinically approved as a chemotherapeutic, has demonstrated broad-spectrum anticancer activity and is currently under investigation for clinical use. (Ferriol, A.; Morán, M. del C. Enhanced Performance of Gelatin 5-Fluorouracil-Containing Nanoparticles against Squamous Cell Carcinoma in Simulated Chronic Wounds Conditions. Mater. Sci. Eng. C 2021, 124, 112073; Kciuk, M.; Giclecińska, A.; Mujwar, S.; Kołat, D.; Kałuzińska-Kołat, Ż.; Celik, I.; Kontek, R. doxorubicin—An Agent with Multiple Mechanisms of Anticancer Activity. Cells 2023, 12 (4), 659; Ishida, J.; Ohtsu, H.; Tachibana, Y.; Nakanishi, Y.; Bastow, K. F.; Nagai, M.; Wang, H.-K.; Itokawa, H.; Lee, K.-H. Antitumor Agents. Part 214: † Synthesis and Evaluation of Curcumin Analogues as Cytotoxic Agents. Bioorg. Med. Chem. 2002, 10 (11), 3481-3487). However, its poor aqueous solubility and rapid degradation in physiological conditions significantly limit its clinical application. (Pan-On, S.; Dilokthornsakul, P.; Tiyaboonchai, W. Trends in Advanced Oral Drug Delivery System for Curcumin: A Systematic Review. J. Controlled Release 2022, 348, 335-345; Liu, T.; Long, T.; Li, H. Curcumin Suppresses the Proliferation of Oral Squamous Cell Carcinoma through a Specificity Protein 1/Nuclear Factor-KB-Dependent Pathway. Exp. Ther. Med. 2021, 21 (3), 202; Mondal, S.; Ghosh, S.; Moulik, S. P. Stability of Curcumin in Different Solvent and Solution Media: UV-Visible and Steady-State Fluorescence Spectral Study. J. Photochem. Photobiol. B 2016, 158, 212-218). Gelatin-based hemostats offer a delivery solution by enhancing solubility and stability of curcumin in situ.

Doxorubicin is one of the most potent FDA-approved chemotherapeutic agents but is limited by dose-dependent toxicity to critical organs including the heart, liver, brain, and kidneys. (Kciuk, M.; Gielecińska, A.; Mujwar, S.; Kołat, D.; Kałuzińska-Kołat, Ż.; Celik, I.; Kontek, R. doxorubicin—An Agent with Multiple Mechanisms of Anticancer Activity. Cells 2023, 12 (4), 659). Localized doxorubicin delivery through gelatin conjugates can reduce systemic exposure and improve tumor specificity. Doxorubicin also contains primary amines capable of covalent conjugation with gelatin via EDC-mediated crosslinking, unlike 5FU and curcumin, which are primarily entrapped within the gelatin matrix.

To evaluate the therapeutic potential of these conjugates for post-surgical treatment of cSCC, the inventors focused on three primary outcomes: (1) successful incorporation of anticancer agents into gelatin-based matrices, (2) sustained release and degradation over a two-week period, and (3) selective cytotoxicity toward cancer cells over healthy cells. Degradation was analyzed using UV-visible spectroscopy, and cytotoxicity was assessed using HEK293 (healthy) and A431 (cSCC) cell lines.

### Materials and Methods

### Materials

Gelatin type B, 2-(N-morpholino) ethanesulfonic acid (MES), 5-fluorouracil (5FU), doxorubicin hydrochloride, curcumin, 1-ethyl-3-(3-dimethylaminopropyl) carbodiimide hydrochloride (EDC), sodium chloride (NaCl), and absolute ethanol were purchased from Fisher Scientific (Waltham, MA). The A431 human cutaneous squamous cell carcinoma cell line and HEK293 human embryonic kidney cells were obtained from the American Type Culture Collection (ATCC, Manassas, VA). Dulbecco's Modified Eagle Medium (DMEM), fetal bovine serum (FBS), antibiotics, and MTT reagents were obtained from standard commercial suppliers.

### Preparation of Drug-Eluting Hemostatic Agents

Drug-eluting gelatin conjugates were synthesized using type B gelatin and 1-ethyl-3-(3-dimethylaminopropyl) carbodiimide (EDC) as the crosslinking agent. Gelatin was prepared as a 20 mg/mL stock solution in 0.05 M MES buffer pH 5.0 with stirring at 50° C. until soluble. To promote dissolution of curcumin prior to crosslinking, curcumin (0.035 g) was added to MES buffer (1 mL) to form a mixture. This mixture was heated at 65° C. for 15 minutes and sonicated in an ice bath for 10 minutes. To prepare the conjugates, 20 mM carboxyl group concentration from the gelatin stock was incubated with either 5-fluorouracil (30.75 mM), doxorubicin (0.1 mM), or curcumin (13.57 mM) under activation by EDC (60 mM) for 2 hours at 50 rpm and 22° C. The product was precipitated under ice-cold absolute ethanol followed by centrifugation at 6000×g, then dissolved in 1.85 mM NaCl for washing, followed by a second round of precipitation. Conjugates were vacuum dried and heated at 80° C. to remove residual ethanol.

Anti-Cancer Drug Release from Gelatin-Based Conjugates

For release profile determination, dried gelatin conjugates loaded with 5-fluorouracil (5FU), doxorubicin, or curcumin were first rehydrated by combining each conjugate with 0.5 mL phosphate-buffered saline (PBS) to form a semisolid matrix having a paste-like consistency. This matrix was then immersed in 1 mL of PBS in a conical vial. The samples were incubated at 37° C. under 100% relative humidity for a total duration of two weeks. At predetermined intervals-24, 48, and 72 hours, followed by 1 week and 2 weeks-1 mL of the PBS was carefully removed and replaced with an equal volume of fresh PBS. All collected release samples were stored at −80° C. until analysis. Prior to quantification, samples were filtered through 0.45 μm syringe filters to eliminate any particulates.

Drug concentrations in the release media were quantified by UV-visible spectroscopy. Measurements were performed at the characteristic wavelengths of each anticancer agent: 295 nm for 5-FU, 489 nm for doxorubicin, and 312 nm for curcumin. Calibration curves generated from standard solutions of each drug in PBS were used to correlate peak absorbance with drug concentration.

### Biocompatibility Analysis of Anti-Cancer Drug-Eluting Gelatin Conjugates

Biocompatibility of gelatin-based conjugates loaded with 5-fluorouracil, doxorubicin, or curcumin was assessed using an indirect cell viability assay with both healthy human embryonic kidney (HEK293) cells and cutaneous squamous cell carcinoma (A431) cells. Cell lines were maintained in complete DMEM supplemented with 10% fetal bovine serum and antibiotics at 37° C. in a humidified incubator with 5% CO₂.

For testing, cells were seeded at 10,000 cells per well in 96-well polystyrene plates and allowed to adhere for 24 hours in complete media. Degradation eluents from loaded and unloaded conjugates were collected at 24, 48, and 72 hours and filtered through 0.2 μm syringe filters.

Additional test conditions included a solution matching the total drug concentration released over the entire two-week degradation period (“matched release”), as well as a dilution series based on the cumulative release profile.

After the initial incubation, media in each well was replaced with 100 μL of filtered test solution. Positive controls consisted of cells cultured in fresh untreated media, while negative controls contained media without cells. All test conditions were applied for 24 hours, after which MTT assays were completed and calculated according to formula (2).

$$Normalized cell viability = \frac{( Abs 600 sample - Abs 600 negative control )}{( Abs 600 positive control - Abs 600 negative control )} \qquad ( 2 )$$

### Results

### Characterization of Anticancer Drug-Eluting Gelatin Conjugates

Gelatin type B was selected as the polymer matrix for all conjugations due to its high density of carboxylate groups, which promotes crosslinking through carbodiimide chemistry. Conjugation was performed in slightly acidic conditions using 0.05 M MES buffer (pH 5.0) to enhance the reactivity of carboxyl groups and facilitate nucleophilic attack by EDC. This environment supported efficient crosslinking and formation of a three-dimensional gelatin network. In general, the EDC performs best in slightly acidic conditions (pH 4-5) but can still crosslink effectively as high as 7.5. The higher the pH, the lower the efficiency of the EDC to crosslink.

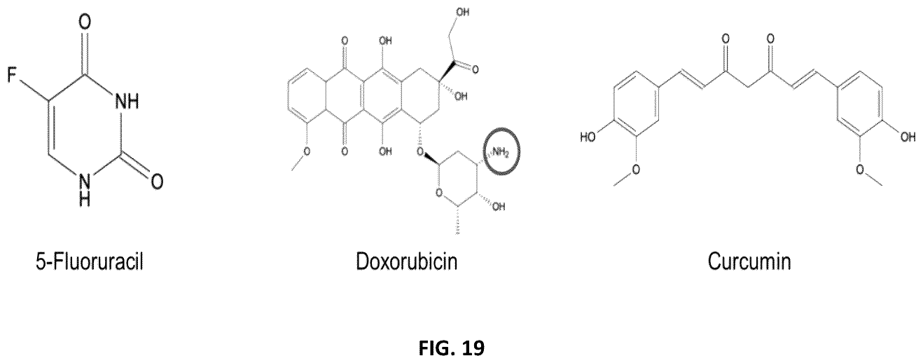

The crosslinking reactions were carried out in the presence of the anticancer agents 5FU, doxorubicin, and curcumin. Due to structural differences, only doxorubicin, containing a primary amine, was capable of forming covalent amide bonds with gelatin. In contrast, 5FU and curcumin were incorporated primarily through physical entrapment within the cross-linked gelatin matrix. The crosslinking process resulted in cage-like structures capable of holding the drug molecules in place while allowing for gradual release over time. This structural distinction, between covalently bound and physically entrapped drug molecules, was expected to influence the degradation and release kinetics of the conjugates. The more available carboxyl or amine groups on the drug, the more ability for the drug to covalently attach to the gelatin thus leading to longer sustained release since the complex would need to degrade for more drug to be released. Here, the 5-FU and curcumin should be released quickly (less than 2 weeks), whereas the release from doxorubicin conjugates should last longer because they are covalently bonded to the gelatin. The representative chemical structures of each of the drugs is shown in FIG. 19.

Anticancer Drug Release from Hemostatic Agents

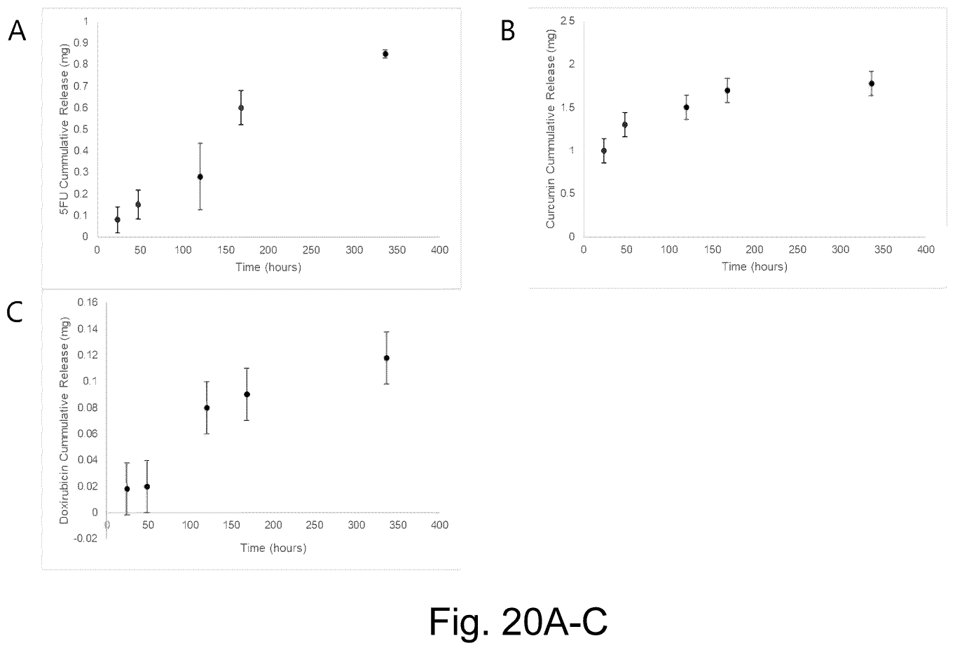

Release profiles of 5-fluorouracil, doxorubicin, and curcumin from gelatin-based conjugates were monitored over a two-week period and are shown in FIG. 20. All drug release studies were conducted under physiological conditions, and UV-visible spectroscopy was used to quantify drug concentrations in the release media at predetermined intervals.

The 5FU conjugates exhibited a consistent and sustained release profile, with approximately 32% of the total loaded drug released over the course of 14 days (FIG. 20A). This linear release suggests gradual diffusion of 5FU from within the gelatin matrix, likely driven by slow degradation of the structure. Notably, the conjugates retained structural integrity by the end of the study, indicating that a substantial amount of drug remained sequestered within the crosslinked network.

Curcumin conjugates also displayed controlled release, with approximately 46% of the total curcumin load released over two weeks (FIG. 20B). Despite curcumin's poor aqueous solubility, sonication and preheating during synthesis appeared to enhance its incorporation and sustained elution. These results support the use of gelatin as an effective delivery matrix for hydrophobic agents like curcumin.

In contrast, doxorubicin conjugates demonstrated highly variable release behavior. Initial release during the first 24 hours was minimal, followed by a sharp increase in release between 24 and 84 hours (FIG. 20C). Beyond this window, release slowed considerably, potentially due to lower concentrations of doxorubicin being used. Doxorubicin release was influenced by its ability to form covalent bonds with gelatin, potentially resulting in both conjugated and physically entrapped drug fractions. Despite optimization attempts, the doxorubicin release profile remained inconsistent, and a distinct cumulative release percentage could not be reliably isolated.

Biocompatibility of Anticancer Agents Released from Hemostatic Agents

While gelatin alone is considered biocompatible, the incorporation of anticancer agents and the use of EDC during crosslinking may alter cytotoxicity. Therefore, cell viability was assessed using degradation media from gelatin-based conjugates loaded with 5-fluorouracil (5FU), doxorubicin, or curcumin, and tested against HEK293 cells. Controls included media from unloaded gelatin conjugates and solutions containing equivalent concentrations of each free drug. In an aspect, according to the literature, exemplary drug concentrations may be between about 0.1 mM to about 0.5 mM.

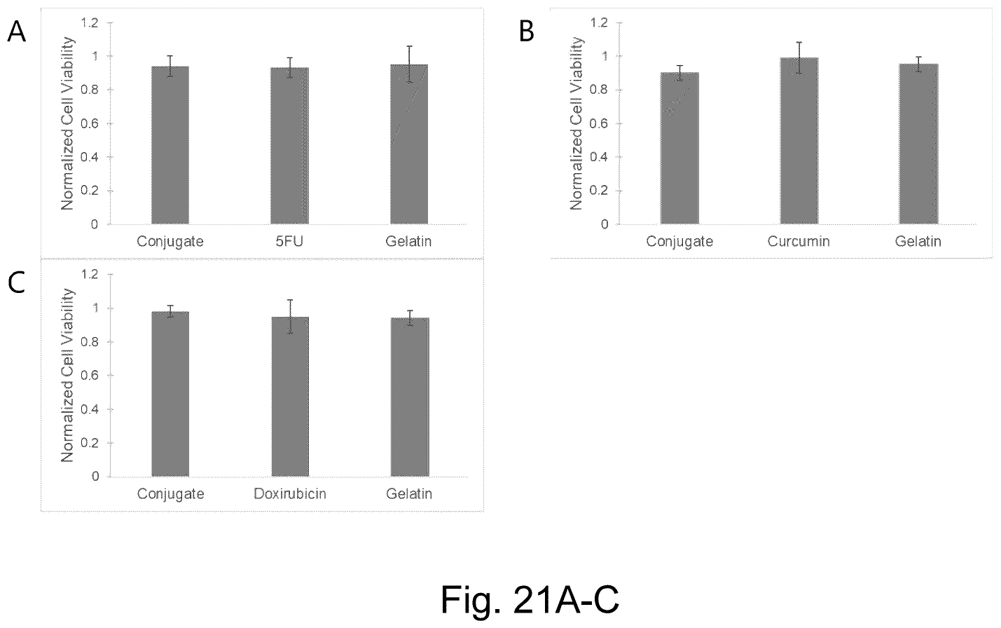

As shown in FIG. 21A-C, all degradation media including those from loaded and unloaded conjugates exhibited low to moderate cytotoxicity toward HEK cells. Among the treatments, the highest level of cytotoxicity was observed in cells exposed to loaded conjugate media, particularly in the 5FU and doxorubicin groups. Importantly, no degradation sample exhibited complete loss of cell viability, indicating that the gelatin-based formulations maintain a favorable biocompatibility profile. These findings support the potential of gelatin as a safe delivery vehicle.

### Example 5—Chitosan Hemostat

### Materials and Methods

### Materials

Chitosan (85% deacetylated), 2-(N-morpholino) ethanesulfonic acid (MES), ampicillin, ciprofloxacin, 1-Ethyl-3-(3-dimethylaminopropyl) carbodiimide (EDC), and absolute ethanol were purchased from Fischer Scientific (Waltham, MA). Tryptic soy agar and broth were purchased from Difco Laboratories (Sparks, MD). HEK-293 fibroblasts and Staphylococcus aureus 25923 were obtained from ATCC (Manassas, VA).

### Synthesis of Chitosan-Based Antibiotic-Eluting Hemostatic Agents

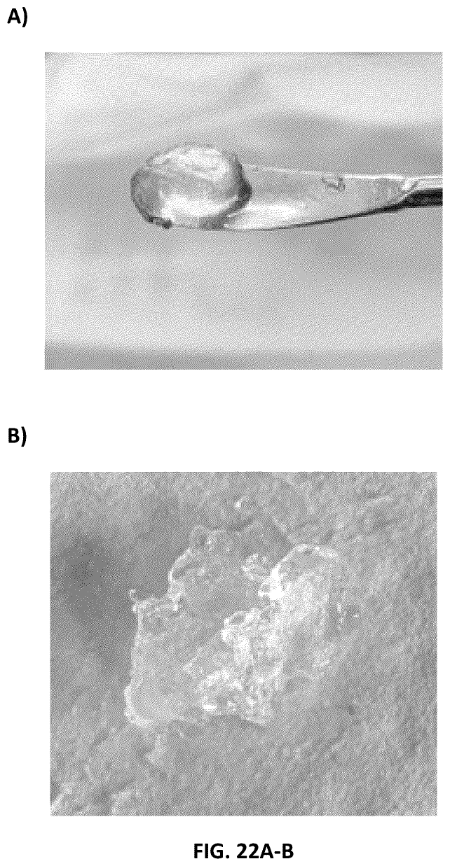

Semi-solid jelly-like chitosan-based hemostatic agents were synthesized by conjugating either ciprofloxacin or ampicillin to chitosan via EDC-mediated crosslinking. Antibiotics were selected based on their clinical relevance in treating oral infections and abscesses. Medium molecular weight chitosan (85% deacetylated) was prepared at a concentration of 2.62 mM in 0.05 M MES buffer (pH 5.0), and solubility was enhanced by bubbling CO₂ through the solution for 20 minutes to slightly lower the pH. Separately, ciprofloxacin (12 mM) or ampicillin (11.5 mM) was dissolved in the same MES buffer. Crosslinking was initiated by combining equal volumes (3 mL each) of chitosan and antibiotic solutions, followed by the addition of freshly prepared EDC (final concentrations 10-50 mM), with 50 mM giving the best results. The reaction mixture was agitated on an orbital shaker at room temperature for 2 hours to facilitate zero-length crosslinking between the carboxylic acid group of the antibiotic and the primary amine groups on chitosan. The products were precipitated under ice-cold absolute ethanol followed by centrifugation at 6000×g to obtain product pellet. Products are jelly-like in appearance. (FIG. 22A). Products were subsequently dried under vacuum and placed on watch glasses in a drying oven overnight at low heat to remove residual ethanol. (FIG. 22B).

### In-Vitro Drug Release

To evaluate antibiotic release, dried conjugates were rehydrated in 1.5 mL of phosphate-buffered saline (PBS, pH 7.4) in conical vials and incubated at 37° C. At predetermined time intervals over a two-week period, 1 mL aliquots of supernatant were collected and replaced with fresh PBS to maintain conditions. UV-Visible spectrophotometry was used to quantify antibiotic concentrations in the collected samples. Calibration curves were generated using known concentrations of ciprofloxacin and ampicillin in PBS (λ_max=270 nm and 213 nm, respectively). The concentrations of antibiotics in the eluents were calculated using the calibration equations and plotted to generate drug release profiles.

### Antibacterial Activity Assay

The antibacterial efficacy of the antibiotic-eluting chitosan conjugates was assessed using the Kirby-Bauer disk diffusion method. Mueller-Hinton agar (MHA) plates were poured and divided into quadrants labeled for (1) filter paper only, (2) chitosan in MES, (3) antibiotic in MES, and (4) crosslinked conjugate. Circular paper disks were soaked in respective solutions and placed in assigned quadrants. Each plate was inoculated with 125 μL of E. coli suspension, spread evenly across the agar surface. Plates were incubated at 37° C. for 24 hours, after which zones of inhibition were measured to evaluate antibacterial activity.

### Results

### Characterization of Antibiotic-Eluting Chitosan Hemostatic Agents

Chitosan, a deacetylated derivative of chitin, was selected as the base polymer due to its abundance of free primary amine groups, which provide reactive sites for covalent crosslinking. Under slightly acidic conditions (pH between 5-7.5) (0.05 M MES buffer, pH 5.0), the carboxylate groups of ciprofloxacin and ampicillin were activated using 1-ethyl-3-(3-dimethylaminopropyl) carbodiimide (EDC), enabling efficient formation of amide bonds with the protonated primary amines of chitosan.

EDC-mediated coupling reactions were conducted in solution-phase, allowing for multiple potential interactions between the antibiotic molecules and the chitosan matrix. The most direct mechanism involves covalent bond formation between the carboxyl group of the antibiotic and an amine group on the chitosan backbone. This results in a stable amide linkage that immobilizes the antibiotic within the matrix. Additionally, physical entrapment of antibiotic molecules within the semi-solid chitosan network likely occurred during the crosslinking process, especially for unreacted or partially reactive antibiotic molecules.

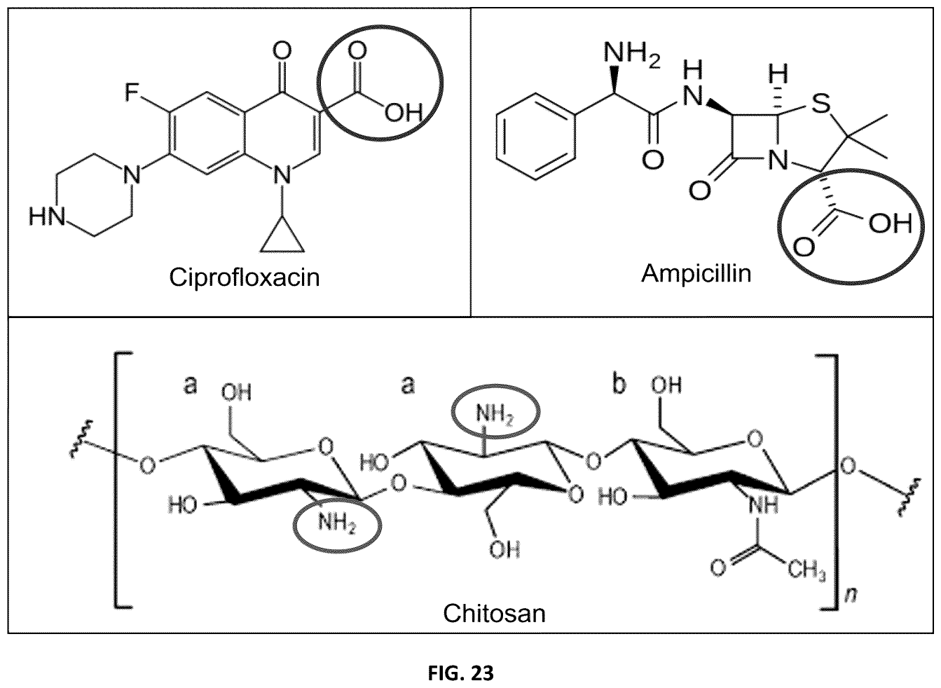

Due to the structural complexity of ciprofloxacin and ampicillin, a variety of binding outcomes are possible (FIG. 23). For example, ciprofloxacin contains a carboxyl group capable of engaging in crosslinking or secondary interactions, while the β-lactam ring of ampicillin may limit the number of reactive conformations, thereby influencing its mode of incorporation.

The combined result of covalent conjugation and physical entrapment is a robust, semi-solid antibiotic-loaded chitosan matrix capable of controlled drug release and targeted antibacterial action in post-surgical environments.

Antibiotic Release from Chitosan Hemostatic Agents

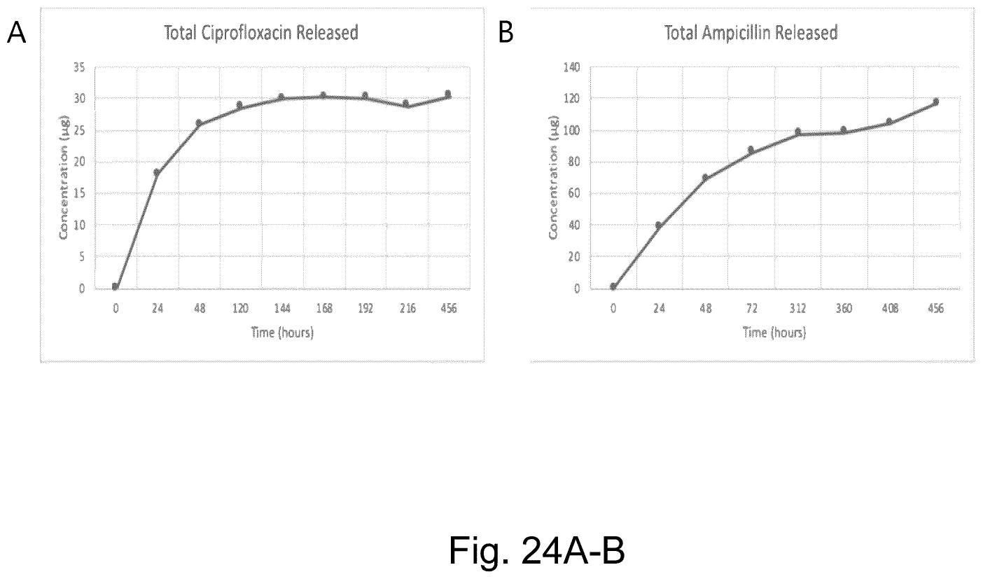

Release profiles of ciprofloxacin- and ampicillin-linked chitosan hemostatic agents, determined by UV-Vis spectrophotometry, are shown in FIG. 24A-B. Ciprofloxacin conjugates exhibited an initial burst release, with approximately half of the total antibiotic load released within the first 24 hours. Following this burst phase, drug release tapered significantly and continued gradually for up to 2 weeks. Ampicillin conjugates showed a similar release pattern with an early burst effect observed at 24 hours, followed by sustained release over 3 weeks. These trends suggest that while both antibiotics were successfully incorporated into the chitosan matrix, the extent of covalent conjugation and physical entrapment may differ, influencing their respective release kinetics. One theory for the consistent and more sustained release of ampicillin may be that ciprofloxacin could be impacted by resonance at the pi bond adjacent the carboxyl thus causing less effective peptide bonding.

Efficacy of Released Antibiotics from Chitosan Hemostatic Agents

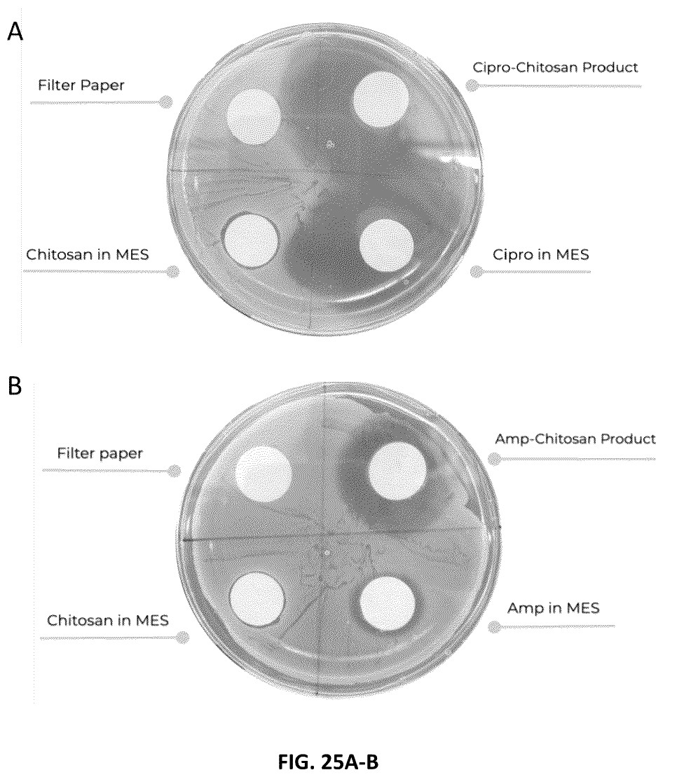

To evaluate the antimicrobial efficacy of ciprofloxacin and ampicillin released from chitosan-based hemostatic agents, modified Kirby-Bauer disk diffusion assays were performed using E. coli as the test organism. Zones of inhibition (ZOI) were observed in plates containing filter paper disks soaked with the released drug, pure antibiotic, and crosslinked conjugates (FIG. 25). In the ciprofloxacin assays, both the pure antibiotic and the ciprofloxacin-chitosan conjugate produced large, clearly defined zones of inhibition, indicating strong and sustained antibacterial activity over the 24-hour incubation period. The comparable size of the inhibition zones supports the conclusion that ciprofloxacin remained structurally intact following its release from the conjugate matrix.

Ampicillin conjugates also demonstrated measurable antibacterial activity against E. coli, though the ZOI was smaller compared to ciprofloxacin. This difference is consistent with the known relative potencies of the two antibiotics against gram-negative bacteria. The presence of inhibition in both conjugate and free-drug controls confirmed that the ampicillin retained therapeutic function after release. These results confirm that the structural integrity and functional efficacy of ciprofloxacin and ampicillin were preserved throughout the synthesis, crosslinking, and release processes. Combined with sustained drug release profiles, these findings support the potential of chitosan-based antibiotic-eluting hemostats for local infection control in post-surgical applications.

### Example 6—Chitosan-Alginate Hemostats

### Materials and Methods

### Materials

Chitosan (medium molecular weight, 85% deacetylated), sodium alginate, 2-(N-morpholino) ethanesulfonic acid (MES), vancomycin hydrochloride, and 1-ethyl-3-(3-dimethylaminopropyl) carbodiimide (EDC) were purchased from Fisher Scientific (Waltham, MA). Sodium chloride (NaCl) was obtained from Sigma-Aldrich (St. Louis, MO).

### Preparation of Chitosan-Alginate-Vancomycin Hemostatic Agents

Chitosan-alginate-vancomycin hemostatic complexes were synthesized via EDC-mediated crosslinking to create a semi-solid jelly-like formulation suitable for wound applications. Medium molecular weight chitosan (85% deacetylated) was prepared at a concentration of 2.62 mM in 0.05 M MES buffer (pH 5.0), and solubility was enhanced by bubbling CO₂ and subjected to mild heating via water bath. Separately, vancomycin (2.06 mM) was dissolved in the same MES buffer. To prepare the conjugates, various volumes of solutions were combined in the presence of EDC (75 mM) and 1 mL of the vancomycin solution as indicated in the table below to obtain varying ratios of chitosan and alginate.

TABLE 1
Complexes synthesized using different
ratios of alginate and chitosan

| | | | Alginate to |
|---|---|---|---|
| | Volume Alginate | Volume Chitosan | Chitosan Volume |
| Complex | (mL) | (mL) | Ratio |
| Complex 1 | 1 | 5 | 1:5 |
| Complex 2 | 2 | 4 | 1:2 |
| Complex 3 | 3 | 3 | 1:1 |
| Complex 4 | 0.5 | 5.5 | 1:11 |

The resulting mixtures were incubated at room temperature on an orbital shaker for 2 hours. To purify the product, the reaction mixture was precipitated with ice-cold absolute ethanol, then centrifuged at 6000×g for 15 minutes at 4° C. to precipitate the polymer complex. The supernatant was decanted, and the pellet was resuspended in 4 mL of 1.85 mM NaCl and briefly heated in a water bath for 2 minutes to aid dissolution. A second round of precipitation was performed with ice-cold ethanol to remove residual EDC and unreacted small molecules to form a semi-solid jelly-like product.

Antibiotic Release from Chitosan-Alginate-Vancomycin Hemostatic Agents

To evaluate vancomycin release profiles from chitosan-alginate hemostatic agents, conjugates were incubated in phosphate-buffered saline (PBS, pH 7.4) at 37° C. For each time point, 1.5 mL of PBS was added to the dried complex in a conical vial. After a 24-hour incubation period, 1 mL of the release medium was removed, transferred to a microcentrifuge tube, and stored at −20° C. The remaining PBS in the vial was replenished with 1 mL of fresh buffer, and the incubation process was repeated for up to one week with daily sampling. After this initial period, samples were collected weekly until the complex dissolved or no further release was detected.

Release samples were analyzed using a UV-visible spectrophotometer (λ=280 nm), and absorbance values were compared against a vancomycin standard curve prepared in PBS to estimate the concentration of antibiotic released at each time point. The resulting drug release profiles were used to evaluate the influence of crosslinking density and polymer composition on vancomycin elution from the hemostatic matrix. Complexes with higher chitosan-to-alginate ratios generally demonstrated improved structural integrity and modest vancomycin release, while formulations with higher alginate content exhibited weaker matrices and limited or undetectable drug elution.

### Fourier Transform Infrared Spectroscopy (FTIR)

FTIR spectroscopy was performed to assess the structural integrity of vancomycin released from chitosan-alginate hemostatic conjugates. Release samples collected at 24, 48, 72, and 96 hours were lyophilized and analyzed using a Nicolet iS10 FTIR spectrometer. Spectra were acquired in the mid-infrared range of 4000-800 cm-1 at a resolution of 4 cm⁻¹.

Spectra were compared to those of pure vancomycin to confirm the presence of characteristic functional groups following release from the polymer matrix. Particular attention was given to peaks corresponding to amide, aromatic, and hydroxyl functionalities to verify that the antibiotic retained its chemical structure after crosslinking and elution. Changes in transmittance over time were also used to infer relative differences in antibiotic concentration across successive release intervals.

### Results

### Characterization of Antibiotic-Eluting Hemostatic Agents

Chitosan and alginate were selected as the primary biopolymers for conjugation due to their complementary properties: chitosan provides rigidity and inherent antimicrobial activity through its primary amine groups, while alginate contributes flexibility, porosity, and fluid absorption capacity through its carboxylate functionalities. Under slightly acidic conditions (0.05 M MES buffer, pH 5.0), EDC-mediated crosslinking enabled formation of amide bonds between the carboxyl groups of alginate and vancomycin and the amine groups of chitosan and vancomycin.

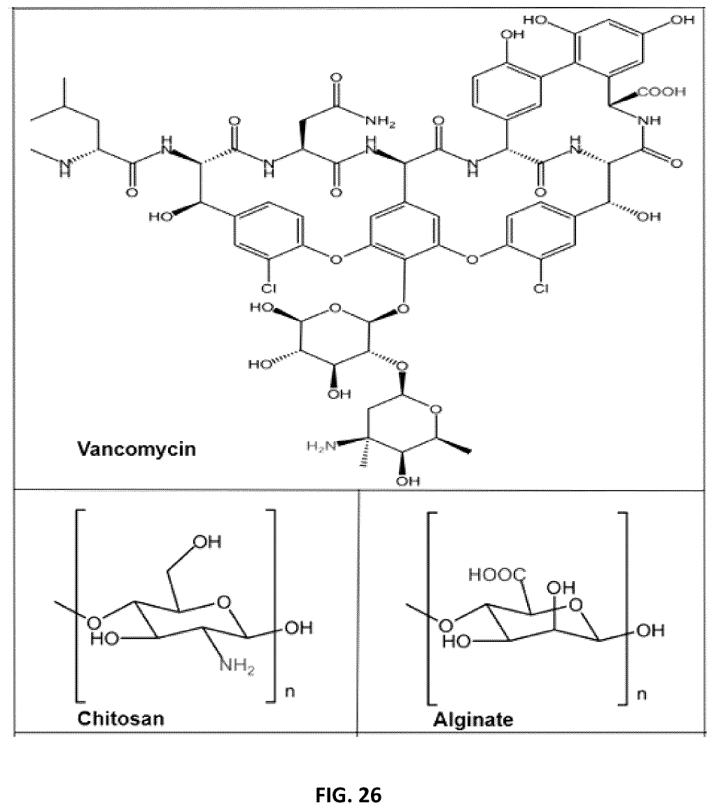

Crosslinking reactions performed in the presence of vancomycin enabled both direct covalent attachment of the antibiotic to the polymer network and potential entrapment of unreacted drug molecules within the semi-solid matrix. The resulting product displayed a semi-solid jelly-like consistency, with moderate structural rigidity suitable for soft tissue application. As illustrated in FIG. 26, multiple reactive sites are available on vancomycin, chitosan, and alginate, allowing for several possible crosslinking outcomes and complex network formation.

Optimization of the alginate-to-chitosan ratio was essential to achieve sufficient gel formation and drug incorporation. Formulations with higher alginate content resulted in weak structural integrity and minimal product formation, while lower alginate ratios promoted stronger crosslinking and greater retention of the hydrogel structure.

Antibiotic Release from Hemostatic Agents

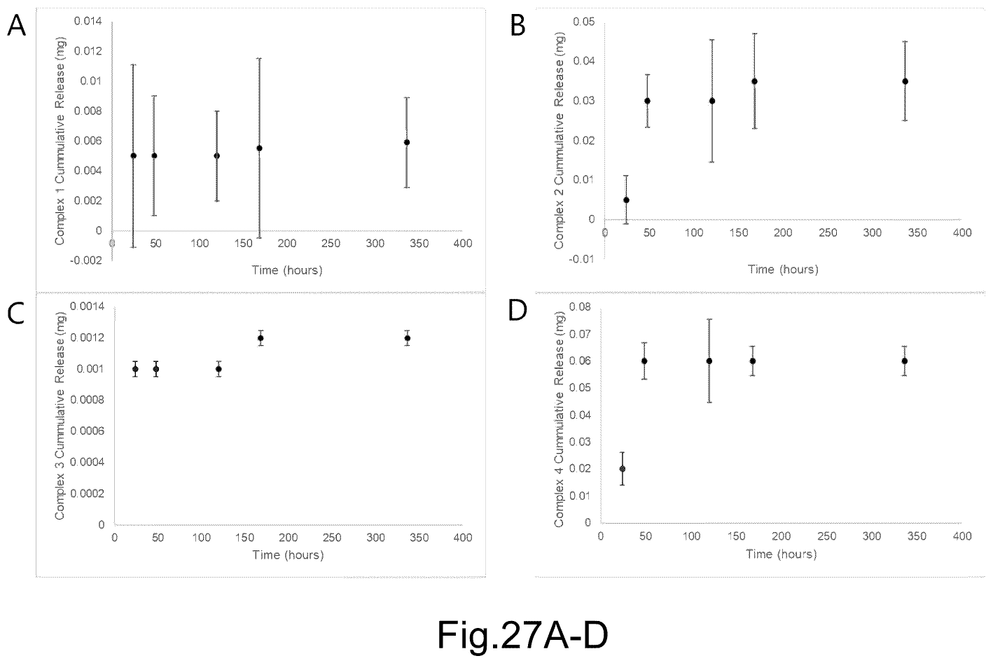

Release profiles of vancomycin from chitosan-alginate hemostatic agents were evaluated over a period of several days using UV-visible spectrophotometry (λ=280 nm) and are shown in FIG. 27. In contrast to traditional gelatin-based systems, the chitosan-alginate conjugates exhibited limited vancomycin release, with most formulations releasing only trace concentrations of the antibiotic during the observation period.

Complex 1, which contained a 1:5 ratio of alginate to chitosan, demonstrated the earliest and highest release of vancomycin, reaching a maximum concentration of approximately 0.0045 mg at 24 hours (FIG. 27A). Despite the observed release, the eluted concentration was substantially below the therapeutic range required for bacterial inhibition. Subsequent time points showed negligible release, likely due to strong crosslinking interactions between vancomycin and the polymer network that restricted drug diffusion.

Complex 2 exhibited a delayed release profile, with detectable vancomycin appearing at 48 hours and reaching a peak concentration of 0.03 mg (FIG. 27B). No further release was detected at later time points, indicating that while some vancomycin elution occurred, the majority remained tightly bound within the matrix.

Complexes 3 and 4 highlighted the importance of polymer ratio on release dynamics. Complex 3, which had the highest alginate content, exhibited no detectable vancomycin release throughout the one-week period (FIG. 27C), likely due to insufficient crosslinking and poor structural integrity. Conversely, Complex 4, which had the lowest alginate-to-chitosan ratio (1:11), produced a slightly higher release profile, with detectable vancomycin at both 24 and 48 hours (FIG. 27D), though the overall concentration remained well below ideal therapeutic thresholds.

Together, these results suggest that while chitosan-alginate matrices can successfully incorporate vancomycin, strong crosslinking interactions, particularly in chitosan-rich formulations, may hinder effective drug elution. However, the chitosan-alginate conjugates degraded substantially slower than the other conjugates tested, lasting longer than the antibiotic-gelatin conjugates by a few weeks. Thus, these conjugates may still be present at least 6 weeks after application, if not longer. Optimization of polymer ratios and crosslinking conditions is performed to achieve sustained therapeutic release suitable for clinical application. Experiments with ratios of 1:10, 1:15, 1:20, 1:25, 1:30, and 1:35 alginate: chitosan are performed to optimize release and crosslinking conditions.

Structure and Integrity of Released Vancomycin from Hemostatic Agents

To determine whether conjugation to the chitosan-alginate matrix affected the chemical structure of vancomycin, FTIR spectroscopy was conducted on release samples collected at multiple time points. Since EDC-mediated crosslinking can potentially modify functional groups, spectra from eluted vancomycin were compared against those of pure vancomycin to evaluate structural preservation.

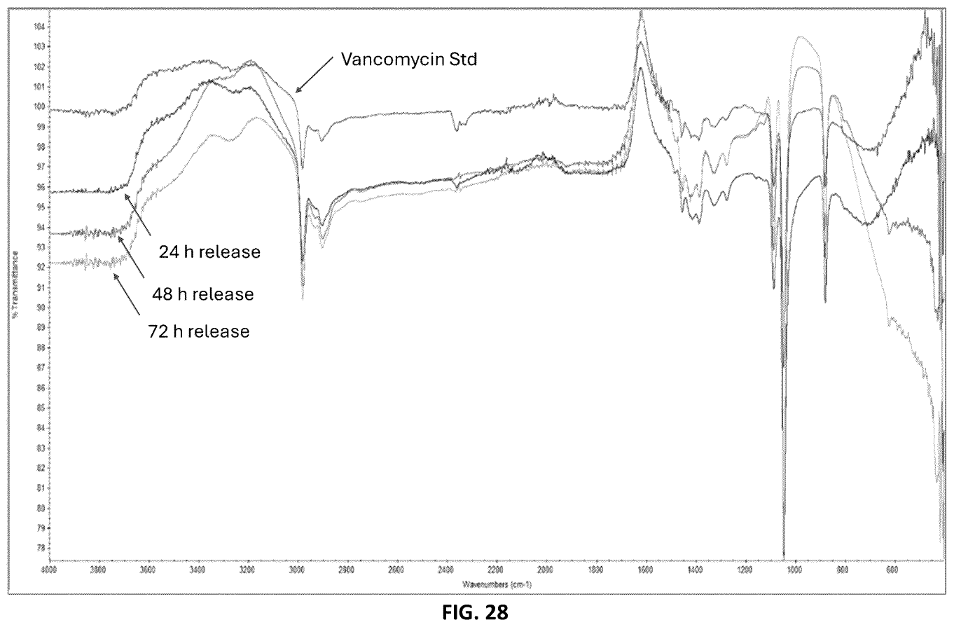

Characteristic absorption peaks corresponding to functional groups in vancomycin particularly those associated with amide, aromatic, and hydroxyl stretches were observed in the release samples. As shown in FIG. 28, the overall spectral patterns remained consistent across samples collected at 24, 48, and 72 hours, confirming that the molecular structure of vancomycin remained largely intact following crosslinking and release from the polymer matrix.

Variations in percent transmittance over time reflected decreasing vancomycin concentrations in the elution media, but no significant shifts or disappearance of key peaks were observed. These findings support the conclusion that vancomycin retained its chemical integrity post-release and remained suitable for antimicrobial activity, provided sufficient concentrations could be achieved.

### Example 7—In Vivo Prophetic Example—Vancomycin in Gelatin Hemostat

A 40-year-old female patient undergoes spinal surgery. An antibiotic-eluting hemostatic agent is prepared using the following steps. Gelatin is prepared as a 20 mg/mL stock solution in 0.05 M MES buffer pH 5.0 with stirring at 50° C. until soluble. To prepare the hemostatic agents, 20 mM carboxyl group concentration from the gelatin stock is incubated with vancomycin (2.07 mM) under activation by EDC (60 mM) for 2 hours at 50 rpm and 22° C. The product is precipitated under ice-cold absolute ethanol followed by centrifugation at 6000×g, then dissolved in 1.85 mM NaCl for washing, followed by a second round of precipitation. Hemostatic agents are vacuum dried and heated at 80° C. to remove residual ethanol. The hemostatic agents are in a powder form after heating. Hemostatic agents are sterilized prior to administration to the patient by gamma or ultraviolet irradiation.

The antibiotic-eluting hemostatic agent containing vancomycin is applied to the surgical site to prevent infection before stitches are used to close the surgical wound site. The antibiotic is both immediately released from the hemostatic agent as well as sustainably released over a 3-week period to prevent infection. The patient's wound shows no signs of infection after 3 weeks. If needed, the hemostatic agent is reapplied. Alternatively, the hemostatic agent could be initially applied after stitches are placed.

### Example 8—In Vivo Prophetic Example—Ciprofloxacin in Gelatin

A 36-year-old male patient undergoes surgery to his leg. An antibiotic-eluting hemostatic agent is prepared using the following steps. Gelatin is prepared as a 20 mg/mL stock solution in 0.05 M MES buffer pH 5.0 with stirring at 50° C. until soluble. To prepare the hemostatic agents, 20 mM carboxyl group concentration from the gelatin stock is incubated with ciprofloxacin (0.19 mM) under activation by EDC (60 mM) for 2 hours at 50 rpm and 22° C. The product is precipitated under ice-cold absolute ethanol followed by centrifugation at 6000×g, then dissolved in 1.85 mM NaCl for washing, followed by a second round of precipitation. Hemostatic agents are vacuum dried and heated at 80° C. to remove residual ethanol. The hemostatic agents are in a powder form after heating. Hemostatic agents are sterilized prior to administration to the patient by gamma or ultraviolet irradiation.

The antibiotic-eluting hemostatic agent containing ciprofloxacin is applied to the surgical site to prevent infection after stitches are placed. The antibiotic is both immediately released from the hemostatic agent as well as sustainably released over a 3-week period to prevent infection. The patient's wound shows no signs of infection after 3 weeks. If needed, the hemostatic agent is reapplied. Alternatively, the hemostatic agent could be initially applied before stitches are used to close the surgical wound site.

### Example 9—In Vivo Prophetic Example—Diclofenac in Gelatin

A 50-year-old male patient undergoes surgery to his arm. An NSAID-eluting hemostatic agent is prepared using the following steps. Gelatin is prepared as a 20 mg/mL stock solution in 0.1 M phosphate buffer having a pH of 7.4 with stirring at 50° C. until soluble. To prepare the hemostatic agents, 20 mM carboxyl group concentration from the gelatin stock is incubated with diclofenac (1 mM) under activation by EDC (60 mM) for 2 hours at 50 rpm and 22° C. The product is precipitated under ice-cold absolute ethanol followed by centrifugation at 6000×g, then dissolved in 1.85 mM NaCl for washing, followed by a second round of precipitation. Hemostatic agents are vacuum dried and heated at 80° C. to remove residual ethanol. The hemostatic agents are in a powder form after heating. Hemostatic agents are sterilized prior to administration to the patient by gamma or ultraviolet irradiation.

The NSAID-eluting hemostatic agent containing diclofenac is applied to the surgical site to prevent inflammation after stitches are placed. The NSAID is both immediately released from the hemostatic agent as well as sustainably released over a 2-week period to prevent infection and decrease pain. The patient's wound shows no signs of inflammation after 2 weeks. If needed, the hemostatic agent is reapplied. Alternatively, the hemostatic agent could be initially applied before stitches are used to close the surgical wound site.

### Example 10—In Vivo Treatment of Skin Cancer with Gelatin Hemostat (Prophetic)

A 45-year-old female patient presents with an irregular mole and is diagnosed with squamous cell carcinoma. The patient undergoes surgery to excise the tumor, and a chemotherapeutic agent-eluting hemostat is prepared via the following steps: Drug-eluting gelatin conjugates were synthesized using type B gelatin and 1-ethyl-3-(3-dimethylaminopropyl) carbodiimide (EDC) as the crosslinking agent. Gelatin having a 20 mM carboxylate concentration was prepared as a 20 mg/mL stock solution in 0.05 M MES buffer pH 5.0 with stirring at 50° C. until soluble. To promote dissolution of curcumin prior to crosslinking, the mixture was heated at 65° C. for 15 minutes and sonicated in an ice bath for 10 minutes. To prepare the conjugates, 20 mM carboxyl group concentration from the gelatin stock was incubated with curcumin (950 mM) under activation by EDC (60 mM) for 2 hours at 50 rpm and 22° C. The product was precipitated under ice-cold absolute ethanol followed by centrifugation at 6000×g, then dissolved in 1.85 mM NaCl for washing, followed by a second round of precipitation. Conjugates were vacuum dried and heated at 80° C. to remove residual ethanol.

The chemotherapeutic agent-eluting hemostatic agent containing curcumin is applied to the surgical site to prevent recurrence while minimizing systemic exposure. The curcumin is sustainably released over a 2-week period. The patient does not have recurrence of the squamous cell carcinoma.

### Example 11—In Vivo Treatment of Post-Operative Oral Surgery Patients with Antibiotic-Eluting Chitosan Hemostat (Prophetic)

A 39-year-old male patient undergoes oral surgery. An antibiotic-eluting hemostatic agent is prepared via the following steps: Medium molecular weight chitosan (85% deacetylated) was prepared at a concentration of 2.62 mM in 0.05 M MES buffer (pH 5.0), and solubility was enhanced by bubbling CO₂ through the solution for 20 minutes to slightly lower the pH. Ampicillin (11.5 mM) was dissolved in the same MES buffer. Crosslinking was initiated by combining equal volumes of chitosan and antibiotic solutions, followed by the addition of freshly prepared EDC (final concentration of 50 mM). The reaction mixture was agitated on an orbital shaker at room temperature for 2 hours to facilitate zero-length crosslinking between the carboxylic acid group of the antibiotic and the primary amine groups on chitosan. The products were precipitated under ice-cold absolute ethanol followed by centrifugation at 6000×g to obtain product pellet. Products were subsequently dried under vacuum and placed on watch glasses in a drying oven overnight at low heat to remove residual ethanol.

The antibiotic-eluting hemostatic agent containing ampicillin is applied to the surgical site to prevent infection after stitches are placed. The antibiotic is both immediately released from the hemostatic agent as well as sustainably released over a 3-week period to prevent infection. The patient's wound shows no signs of infection after 3 weeks. If needed, the hemostatic agent is reapplied. Alternatively, the hemostatic agent can be initially applied before stitches are used to close the surgical wound site.

### CONCLUSION

The inventors have developed a novel methodology for the controlled release of chemotherapeutic agents, antibiotics and NSAIDs from active agent-eluting hemostatic agents. Through EDC-catalyzed conjugation, chemotherapeutic agents, antibiotics and NSAIDs were successfully entrapped within gelatinous “cages” as well as directly conjugated to a polymer such as gelatin. The active agent-eluting hemostatic agents produced herein can be instrumental in antimicrobial prophylaxis and reducing inflammation and pain following surgery to help lower rates of surgical site infections and reduce unnecessary systemic administration of antibiotics or anti-inflammatories. This methodology can be further utilized to develop an assortment of products including bandages, suture replacements, and additional hemostatic agents which require a slow release of drugs that exhibit the required functional groups.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. An active agent eluting hemostatic agent capable of immediate and sustained release of the active agent comprising:
a first amount of at least one active agent, having at least one amine or carboxylate group in its structure, directly conjugated to a crosslinked polymer; and
a second amount of the at least one active agent entrapped within cages formed in the crosslinked polymer wherein the polymer is crosslinked to itself via peptide bonds to form the cages;
wherein the crosslinked polymer is gelatin or chitosan;
wherein the active agent eluting hemostatic agent does not contain any additional polymers;
wherein the sustained release occurs over at least 2 weeks;
wherein the hemostatic agent is not a microparticle or nanoparticle.

2. The active agent eluting hemostatic agent of claim 1, wherein the polymer is crosslinked by the crosslinking agent selected from the group consisting of 1-ethyl-3-(3-dimethylaminopropyl) carbodiimide (EDC) and dicyclohexylcarbodiimide (DCC), and carbonyldiimidazole (CDI).

3. The active agent eluting hemostatic agent of claim 1, wherein the at least one active agent is an anesthetic, an antimicrobial, or a chemotherapeutic agent.

4. The active agent eluting hemostatic agent of claim 3, wherein the antimicrobial is an antibiotic comprising vancomycin, daptomycin, ciprofloxacin, ampicillin, or amoxicillin.

5. The active agent eluting hemostatic agent of claim 3, wherein the chemotherapeutic agent comprises 5-fluorouracil (5FU), doxorubicin, or curcumin.

6. The active agent eluting hemostatic agent of claim 3, wherein the anesthetic is a non-steroidal anti-inflammatory drug (NSAID).

7. The active agent eluting hemostatic agent of claim 6, wherein the NSAID comprises aspirin, ibuprofen, naproxen and naproxen sodium, diclofenac, oxaprozin, etodolac, indomethacin, ketorolac, or vimovo.

8. A method of delivering a chemotherapeutic agent to a patient diagnosed with a skin cancer comprising:
applying a therapeutically effective amount of a chemotherapeutic agent eluting hemostatic agent to a tumor site of the skin cancer on the patient, the chemotherapeutic agent eluting hemostatic agent comprising:
at least one chemotherapeutic agent, having at least one amine or carboxylate group in its structure, entrapped within cages formed in a crosslinked gelatin;
wherein the hemostatic agent releases the at least one chemotherapeutic agent by controlled sustained release over at least two weeks.

9. The method of claim 8, further comprising excising the tumor prior to applying the therapeutically effective amount of the chemotherapeutic agent eluting hemostatic agent to the tumor site.

10. The method of claim 8, wherein the at least one chemotherapeutic agent is 5-fluorouracil (5FU), doxorubicin, or curcumin.

11. The method of claim 8, wherein the skin cancer is squamous cell carcinoma (SCC), basal cell carcinoma (BCC), melanoma, or Merkel-cell carcinoma.

12. The method of claim 11, wherein the skin cancer is SCC.

13. The method of claim 8, wherein the gelatin is crosslinked by a chemical crosslinker selected from the group consisting of 1-ethyl-3-(3-dimethylaminopropyl) carbodiimide (EDC), dicyclohexylcarbodiimide (DCC), and carbonyldiimidazole (CDI).

14. The method of claim 13, wherein the chemical crosslinker is EDC at a concentration between about 20 mM to about 60 mM.

15. A method of inhibiting an infection in a surgical site of a patient comprising:
applying a therapeutically effective amount of an antimicrobial-eluting hemostatic agent to the surgical site, the antimicrobial eluting hemostatic agent comprising:
a first amount of at least one antimicrobial, having at least one amine or carboxylate group in its structure, conjugated directly to a crosslinked chitosan matrix via covalent bond; and
a second amount of the at least one antimicrobial entrapped within cages formed in the crosslinked chitosan matrix;
wherein the hemostatic agent releases a portion of the at least one antimicrobial immediately and releases remaining portion by controlled sustained release over at least two weeks;
wherein the hemostatic agent is not a microparticle or nanoparticle.

16. The method of claim 15, wherein the antimicrobial eluting hemostatic agent is produced by a process comprising:
preparing a chitosan solution in which solubility is enhanced to lower pH of the chitosan solution;
dissolving the antimicrobial in buffer to form an antimicrobial solution;
combining equal amounts of the chitosan solution and the antimicrobial solution to form a mixture;
subsequently adding an amount of a crosslinking agent to the mixture;
agitating the mixture to facilitate zero-length crosslinking; and
precipitating, centrifuging, and drying the mixture to form the antimicrobial eluting hemostatic agent.

17. The method of claim 15, wherein the surgical site is an oral surgical site.

18. The method of claim 15, wherein the hemostatic agent further comprising an amount of alginate to form a chitosan-alginate matrix wherein the alginate is present in the chitosan-alginate matrix in an amount less than an amount of chitosan.

19. The method of claim 15, wherein the chitosan is crosslinked by a chemical crosslinker selected f from the group consisting of 1-ethyl-3-(3-dimethylaminopropyl) carbodiimide (EDC), dicyclohexylcarbodiimide (DCC), and carbonyldiimidazole (CDI).

20. The method of claim 15, wherein the antimicrobial is vancomycin, daptomycin, ciprofloxacin, ampicillin, or amoxicillin.
